SEQUENCE LISTING (52 sequences)

SEQ ID NO: 1 - DNA, 19 bp, Artificial Sequence
  primer for CALB2
ctccaggaatacacccaaa

SEQ ID NO: 2 - DNA, 20 bp, Artificial Sequence
  primer for CALB2
cagctcatgctcgtcaatgt

SEQ ID NO: 3 - DNA, 20 bp, Artificial Sequence
  primer for Dlx2
gcacatgggttcctaccagt

SEQ ID NO: 4 - DNA, 20 bp, Artificial Sequence
  primer for Dlx2
tccttctcaggctcgttgtt

SEQ ID NO: 5 - DNA, 20 bp, Artificial Sequence
  primer for Dlx5
ccaaccagccagagaaagaa

SEQ ID NO: 6 - DNA, 20 bp, Artificial Sequence
  primer for Dlx5
gcaaggcgaggtactgagtc

SEQ ID NO: 7 - DNA, 21 bp, Artificial Sequence
  primer for Emx1
aagcgcggctttaccatagag

SEQ ID NO: 8 - DNA, 19 bp, Artificial Sequence
  primer for Emx1
gctggggtgagggtagttg

SEQ ID NO: 9 - DNA, 21 bp, Artificial Sequence
  primer for FoxG1
agaagaacggcaagtacgaga

SEQ ID NO: 10 - DNA, 21 bp, Artificial Sequence
  primer for FoxG1
tgttgagggacagattgtggc

SEQ ID NO: 11 - DNA, 21 bp, Artificial Sequence
  primer for GABRA1
ggattgggagagcgtgtaacc

SEQ ID NO: 12 - DNA, 19 bp, Artificial Sequence
  primer for GABRA1
tgaaacgggtccgaaactg

SEQ ID NO: 13 - DNA, 20 bp, Artificial Sequence
  primer for GABRA2
gttcaagctgaatgcccaat

SEQ ID NO: 14 - DNA, 20 bp, Artificial Sequence
  primer for GABRA2
acctagagccatcaggagca

SEQ ID NO: 15 - DNA, 19 bp, Artificial Sequence
  primer for GABRA5
atcttggatgggctcttgg

SEQ ID NO: 16 - DNA, 20 bp, Artificial Sequence
  primer for GABRA5
tgtactccatttccgtgtcg

SEQ ID NO: 17 - DNA, 20 bp, Artificial Sequence
  primer for GAD65
ggtggctccagtgattaaag

SEQ ID NO: 18 - DNA, 20 bp, Artificial Sequence
  primer for GAD65
tgtccaaggcgttctatttc

SEQ ID NO: 19 - DNA, 19 bp, Artificial Sequence
  primer for GAD67
aggcaatcctccaagaacc

SEQ ID NO: 20 - DNA, 19 bp, Artificial Sequence
  primer for GAD67
tgaaagtccagcaccttgg

SEQ ID NO: 21 - DNA, 20 bp, Artificial Sequence
  primer for GFAP
caacctgcagattcgagaaa

SEQ ID NO: 22 - DNA, 20 bp, Artificial Sequence
  primer for GFAP
gtcctgcctcacatcacatc

SEQ ID NO: 23 - DNA, 21 bp, Artificial Sequence
  primer for Gli3
tggttacatggagccccacta

SEQ ID NO: 24 - DNA, 23 bp, Artificial Sequence
  primer for Gli3
gaatcggagatggatcgtaatgg

SEQ ID NO: 25 - DNA, 20 bp, Artificial Sequence
  primer for LHX6
gggcgcgtcataaaaagcac

SEQ ID NO: 26 - DNA, 20 bp, Artificial Sequence
  primer for LHX6
tgaacggggtgtagtggatg

SEQ ID NO: 27 - DNA, 23 bp, Artificial Sequence
  primer for Map2
cgctcagacacccttcagataac

SEQ ID NO: 28 - DNA, 23 bp, Artificial Sequence
  primer for Map2
aaatcatcctcgatggtcacaac

SEQ ID NO: 29 - DNA, 19 bp, Artificial Sequence
  primer for Mash1
tgcactccaatcattcacg

SEQ ID NO: 30 - DNA, 20 bp, Artificial Sequence
  primer for Mash1
gtgcgtgttagaggtgatgg

SEQ ID NO: 31 - DNA, 21 bp, Artificial Sequence
  primer for Musashi
ttcgggtttgtcacgtttgag

SEQ ID NO: 32 - DNA, 20 bp, Artificial Sequence
  primer for Musashi
ggcctgtataactccggctg

SEQ ID NO: 33 - DNA, 21 bp, Artificial Sequence
  primer for Nestin
cacctgtgccagcctttctta

SEQ ID NO: 34 - DNA, 20 bp, Artificial Sequence
  primer for Nestin
tttcctcccaccctgtgtct

SEQ ID NO: 35 - DNA, 19 bp, Artificial Sequence
  primer for NKX2.1
gtgagcaagaacatggccc

SEQ ID NO: 36 - DNA, 20 bp, Artificial Sequence
  primer for NKX2.1
aaccagatcttgacctgcgt

SEQ ID NO: 37 - DNA, 20 bp, Artificial Sequence
  primer for Olig2
gctgcgacgactatcttccc

SEQ ID NO: 38 - DNA, 19 bp, Artificial Sequence
  primer for Olig2
gcctcctagcttgtcccca

SEQ ID NO: 39 - DNA, 21 bp, Artificial Sequence
  primer for Pax6
aggtattacgagactggctcc

SEQ ID NO: 40 - DNA, 22 bp, Artificial Sequence
  primer for Pax6
tcccgcttatactgggctattt

SEQ ID NO: 41 - DNA, 20 bp, Artificial Sequence
  primer for SCN5A
ggatcgagaccatgtgggac

SEQ ID NO: 42 - DNA, 20 bp, Artificial Sequence
  primer for SCN5A
gctgtgaggttgtctgcact

SEQ ID NO: 43 - DNA, 21 bp, Artificial Sequence
  primer for Sox1
agatgccacactcggagatca

SEQ ID NO: 44 - DNA, 24 bp, Artificial Sequence
  primer for Sox1
gagtacttgtcctccttgagcagc

SEQ ID NO: 45 - DNA, 20 bp, Artificial Sequence
  primer for Sox2
agtctccaagcgacgaaaaa

SEQ ID NO: 46 - DNA, 20 bp, Artificial Sequence
  primer for Sox2
gcaagaagcctctccttgaa

SEQ ID NO: 47 - DNA, 20 bp, Artificial Sequence
  primer for TuJ1
ggcctttggacatctcttca

SEQ ID NO: 48 - DNA, 20 bp, Artificial Sequence
  primer for TuJ1
atactcctcacgcaccttgc

SEQ ID NO: 49 - DNA, 21 bp, Artificial Sequence
  primer for Vimentin
agaactttgccgttgaagctg

SEQ ID NO: 50 - DNA, 23 bp, Artificial Sequence
  primer for Vimentin
ccagagggagtgaatccagatta

SEQ ID NO: 51 - DNA, 20 bp, Artificial Sequence
  primer for GAPDH
gtcagtggtggacctgacct

SEQ ID NO: 52 - DNA, 20 bp, Artificial Sequence
  primer for GAPDH
caccaccctgttgctgtagc

(19) United States
(12) Patent Application Publication
(10) Pub. No.: US 2020/0002679 A1
(43) Pub. Date: Jan. 2, 2020

(54) COMPOSITION FOR CELL REGENERATION COMPRISING CELLS HYPERSECRETING GROWTH FACTORS, AND AT LEAST ONE OF NEURAL STEM CELLS, NEURONS AND GABAERGIC NEURONS

(71) Applicant: KCELLBIO BANKING CO., LTD., Seoul (KR)

(72) Inventors: Mi Sook CHANG, Seoul (KR); Ji Hye PARK, Seoul (KR)

(21) Appl. No.: 16/432,341

(22) Filed: Jun. 5, 2019

Related U.S. Application Data
(60) Provisional application No. 62/680,773, filed on Jun. 5, 2018.

(51) Int. Cl. C12N 5/0775; A61K 35/30; C12N 5/0793

(52) U.S. Cl. CPC C12N 5/0667; A61K 35/30; C12N 5/0619; C12N 2506/1384; C12N 2501/165; C12N 2506/1353; C12N 2501/12

(57) ABSTRACT

A cell composition includes neural stem cells and neural stem cells derived from mesenchymal stem cells, and at least one selected from the group consisting of immature neural stem cells and mesenchymal stem cells. The number of the neural stem cells is at least 70% of the number of the cells included in the cell composition, and the total number of the cells of the immature neural stem cells and the mesenchymal stem cells is at most 30% of the number of the cells included in the cell composition. The cell composition has a very low probability of being developed into a cancer cell and thus can be used as a cell therapeutic.

### FIELD OF THE INVENTION

The present invention disclosed herein relates to a composition for cell regeneration which includes cells that hypersecrete a growth factor, and at least one among neural stem cells, neurons, and GABAergic neurons and/or a cell composition which includes neural stem cells, neurons, and GABAergic neurons.

### BACKGROUND ART

There is still no clear treatment for nervous system injuries and neurodegenerative diseases, and thus, these diseases remain as major issues for clinicians and scientists to solve. Stem cells have self-renewal capacity and the ability to differentiate into various cell lineages and are considered to be an effective source for cell therapy. Human pluripotent stem cells, which are included in human embryonic stem cells (ESCs) and induced pluripotent stem cells (iPSCs), are potent candidates that can be applied to regeneration regenerative therapy and are useful in the biomedical field and the clinical research field (e.g., various metabolic diseases, genetic diseases, degenerative diseases, etc.). However, there is an ethical issue associated with ESCs and iPSCs have safety issues associated with exogenous reprogramming factors which may cause the activation of oncogenic pathways and technical issues (e.g., slow reprogramming process, low efficiency, etc.), and thus there are still hurdles to overcome for the clinical application of iPSCs.

As such, adult mesenchymal stem cells have problems in that they have a lower cell proliferation capacity and a lower differentiation potency than ESCs and iPSCs, however, adult mesenchymal stem cells have advantages in that they have a self-adaptability, high obtainability, strong proliferation capacity, etc. and are thus spotlighted as a more desirable source for regenerative medicine.

Due to these advantages, attempts have been made to use adult mesenchymal stem cells as a source of various cells. In particular, various efforts have been made for the differentiation of adult mesenchymal stem cells into neural stem cells, neurons, or GABAergic neurons. However, there has not been known any cell composition which includes cells that are converted to neural stem cells, neurons, or GABAergic neurons at a high conversion rate.

### SUMMARY OF THE INVENTION

To solve the above-mentioned limitations, the present invention provides a composition for cell regeneration which can promote cell growth in vivo or in vitro.

The present invention also provides a cell composition for cell regeneration which can prevent, improve, or treat neurological disorders and/or a cell composition which includes neural stem cells, neurons, or GABAergic neurons.

In addition, the present invention provides a cell composition for cell regeneration and/or a cell composition which includes neural stem cells, neurons, or GABAergic neurons, which can be used as a therapeutic agent for neurological disorders, even if cells at the intermediate stage of conversion are included.

Further, the present invention provides a composition for cell regeneration and/or a cell composition which includes neural stem cells, neurons, or GABAergic neurons, which has a low probability of causing side effects when embryonic stem cells or induced pluripotent stem cells are applied in vivo.

Additionally, the present invention provides a composition for cell regeneration which can be utilized as a cosmetic composition.

Furthermore, the present invention provides a composition for cell regeneration which has excellent bioadaptability.

In addition, the present invention provides a cell composition which can be utilized as an excellent source for neural stem cells, neurons, or GABAergic neurons.

The present invention relates to a cell composition which includes: neural stem cells; and

neural stem cells derived from mesenchymal stem cells; and

at least one selected from the group consisting of immature neural stem cells and mesenchymal stem cells;

wherein the number of the neural stem cells is at least 70% of the number of the cells included in the cell composition, and the total number of the cells of the immature neural stem cells and the mesenchymal stem cells is at most 30% of the number of the cells included in the cell composition.

The neural stem cells may express at least one selected from the group consisting of Nestin, Sox2, Sox1, Musashi-1, FoxG1, Nkx2.1, Pax6, Gli3, Vimentin, TUj1, Emx1, Olg2, and Ascl1.

Additionally, the cell composition may further include neurons.

Further, the cell composition may further include GABAergic neurons.

The neural stem cells may undergo a symmetric or asymmetric division.

Meanwhile, in the cell composition, the mesenchymal stem cells may be adipose tissue-derived or bone marrow-derived.

In particular, the neural stem cells, which are differentiated from neural stem cells and mesenchymal stem cells, may express at least one selected from the group consisting of Nestin, Sox2, Sox1, Musashi-1, FoxG1, Nkx2.1, Pax6, Gli3, Vimentin, Tuj1, Emx1, Olig2, and Ascl1.

Additionally, the GABAergic neurons may express at least one selected from the group consisting of MAP2, Dlx2, Dlx5, GFAP, CALB2, GABRA1, GABRA2, GABRA5, GAD65, GAD67, PSD95, SYP, and NF-M.

The cell composition of the present invention may further include a cell growth factor, and the cell growth factor is characterized to be epidermal growth factor, fibroblast growth factor, vascular endothelial growth factor (VEGF), hepatocyte growth factor (HGF), nerve growth factor (NGF), glial cell line-derived neurotrophic factor (GDNF), and platelet-derived growth factor (PDGF).

The present invention provides neurons differentiated from mesenchymal stem cells, and the neurons express at least one selected from the group consisting of Tuj1, MAP2, FoxG1, Nkx2.1, Pax6, Dlx2, Dlx5, Lhx6, GAD67, SCN5A, S100, Olg2, and NeuN.

The present invention provides GABAergic neurons differentiated from mesenchymal stem cells, and the GABAergic neurons express at least one selected from the group consisting of NKX2.1, LHX6, TuJ1, MAP2, MGE, Dlx2, Dlx5, GABRA1, GABRA2, GABRA5, CALB2, GAD65, GAD67, PSD95, NF-M, and SYP. In particular, the GABAergic neurons may have a radial neurite and exhibit a spontaneous inhibitory post-synaptic current (IPSC) when treated with a glutamic acid receptor blocker.

Meanwhile, the mesenchymal stem cells may be adipose tissue-derived mesenchymal stem cells, which are neurons.

Additionally, the mesenchymal stem cells may be adipose tissue-derived mesenchymal stem cells, which are GABAergic neurons.

Additionally, the present invention provides a composition for cell regeneration which includes at least one selected from neural stem cells, neurons and GABAergic neurons, and cells that hypersecrete a growth factor.

In the composition for cell regeneration, the neural stem cells, neurons, and GABAergic neurons may be differentiated from adipose tissue-derived mesenchymal stem cells. Additionally, the cells that hypersecrete a growth factor may be differentiated from bone marrow-derived mesenchymal stem cells.

Additionally, the composition for cell regeneration may further include cells which express a marker that is expressed at least one cell selected from the group consisting of astrocytes and oligodendrocytes.

In particular, the neural stem cells may undergo a symmetric division, and may express at least one selected from the group consisting of Nestin, Sox2, Sox1, Musashi-1, FoxG1, Nkx2.1, Pax6, Gli3, Vimentin, Tuj1, Emx1, and Ascl1.

The neurons may express at least one selected from the group consisting of Tuj1, MAP2, FoxG1, Nkx2.1, Pax6, Dlx2, Dlx5, Lhx6, GAD67, SCN5A, GFAP, S100, Olig2, and NeuN.

The GABAergic neurons may express at least one selected from the group consisting of NKX2.1, LHX6, TuJ1, MAP2, MGE, Dlx2, Dlx5, GABRA1, GABRA2, GABRA5, CALB2, GAD65, GAD67, PSD95, NF-M, and SYP.

Additionally, the GABAergic neurons may have a radial neurite and exhibit a spontaneous inhibitory post-synaptic current (IPSC) when treated with a glutamic acid receptor blocker.

In the composition, the cells that hypersecrete a growth factor may express at least one selected from the group consisting of GFAP, P0, S100, CNPase, and p75NTR.

Additionally, the growth factor may be at least one selected from the group consisting of hepatocyte growth factor (HGF), vascular endothelial growth factor (VEGF), insulin-like growth factor binding protein-1 (IGFBP-1), IGFBP-2, IGFBP-4, IGFBP-6, and stem cell factor (SCF), VEGF, and HGF.

Additionally, the growth factor may be at least one selected from the group consisting of VEGF and HGF.

The composition for cell regeneration of the present invention may further include at least one selected from the group consisting of nerve growth factor (NGF), brain-derived neurotrophic factor (BDNF), neurotrophin-3 (NT-3), neurotrophin-4/5 (NT-4/5), neurotrophin-6 (NT-6), basic-FGF, acidic-FGF, FGF-5, epidermal growth factor (EGF), hepatocyte growth factor (HGF), insulin, insulin-like growth factor-1 (IGF-1), insulin-like growth factor-2 (IGF-2), glia cell line-derived neurotrophic factor (GDNF), TGF-β2, TGF-β3, interleukin-6 (IL-6), ciliary neurotrophic factor (CNTF), and LIF.

Meanwhile, the present invention provides a pharmaceutical composition for improving or treating ischemic diseases containing a composition for cell regeneration. In particular, the ischemic disease may be selected from the group consisting of trauma, rejection during transplantation, ischemic cerebrovascular disorder, ischemic renal disease, ischemic lung disease, ischemic disease associated with infectious disease, ischemic disease of limbs, and ischemic heart disease.

Additionally, the present invention provides a pharmaceutical composition for improving or treating neurological disorders containing a composition for cell regeneration. In particular, the neurological disorders may be selected from the group consisting of strokes, Parkinson's disease, Alzheimer's disease, Pick's disease, Huntington's disease, amyotrophic lateral sclerosis, traumatic central nervous system diseases, and spinal cord injury disease.

The cell composition of the present invention can improve growth, survival rate, differentiation rate, etc. of the cells at the injected site in vivo.

The cell composition of the present invention can be utilized as an excellent source for neural stem cells, neurons, or GABAergic neurons.

The cell composition, neural stem cells, neurons, or GABAergic neurons of the present invention have a low probability of causing cytotoxicity when applied in vivo and thus can be used as a safe cell therapeutic.

The cell composition, neural stem cells, neurons, or GABAergic neurons of the present invention can effectively prevent, improve, or treat neurological disorders.

The cell composition of the present invention can be applied for the treatment of neurological disorders without the need to remove the cells which have not been converted to final neural stem cells, neurons, or GABAergic neurons.

The cell composition, neural stem cells, neurons, or GABAergic neurons of the present invention have a very low probability of being developed into cancer cells, and thus can be used as a cell therapeutic.

The cell composition, neural stem cells, neurons, or GABAergic neurons of the present invention can have more excellent differentiation potency, survival rate, settlement ability, expression of markers, etc. compared to the neural stem cell, neurons, or GABAergic neurons in vivo.

The cell composition of the present invention can be utilized as a cosmetic composition as well as a therapeutic agent for neurological disorders.

The composition for cell regeneration of the present invention can promote the growth of cells in vivo or in vitro.

The composition for cell regeneration of the present invention can exhibit the effects of increasing the growth of damaged neurons, nerve fibers, etc., and inhibition of cell death.

The composition for cell regeneration of the present invention can be used as a therapeutic agent for neurological disorders even if cells at the intermediate stage of conversion are included therein.

The composition for cell regeneration of the present invention can prevent, improve, or treat various neurological disorders, ischemic diseases, etc.

The composition for cell regeneration of the present invention has a low probability of causing side effects when embryonic stem cells (ESCs) or induced pluripotent stem cells (IPSCs) are applied in vivo.

The composition for cell regeneration of the present invention can be utilized as a cosmetic composition.

The composition for cell regeneration of the present invention provides a composition for cell regeneration with excellent bioadaptability.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIGS. 1A to 1D shows drawings illustrating optimization of a cross-differentiation protocol for neural stem cells using a small molecule inhibitor (SMI):

FIG. 1A: A diagram illustrating a process of inducing adipocyte-derived mesenchymal stem cells into neural stem cells;

FIG. 1B: Scale bar: 100 μm;

FIG. 1C: A diagram illustrating the results in which the characteristics of induced neural stem cells are analyzed through real-time PCR. The vertical axis represents a relative level of gene expression;

*P<0.05, **P<0.01, and ***P<0.001 significance probabilities are values compared with that of adipocyte-derived mesenchymal stem cells, and ^{†}P<0.05, ^{††}P<0.01, and ^{†††}P<0.001 significance probabilities are values compared with the group which is not treated with a small molecule inhibitor; and

FIG. 1D: Graphs illustrating the quantification of induced neural stem cells (iNSCs) by flow cytometry of neural cell adhesion molecule (NCAM) using a fluorescent flow cytometer (FACS Caliber);

FIGS. 2A to 2B shows drawings illustrating the characteristics of induced neural stem cells (iNSCs) which were subject to cross-differentiation by treatment with a small molecule inhibitor:

FIG. 2A: Images illustrating the detection of induced neural stem cells (iNSCs) which express both Nestin and Sox2 via fluorescent immunocytochemistry (ICC) analysis;

FIG. 2B: Graphs illustrating the changes in expression levels of neural stem cells and early neuronal markers (Sox1, Sox2, Nestin, Musashi-1, FoxG1, Nkx2.1, Pax6, Gli3, Vimentin, Tuj1, and Emx1) using real-time PCR analysis according to the cross-differentiation process of neural stem cells using real-time PCR analysis. The vertical axis represents a relative level of gene expression;

*P<0.05, **P<0.01, ***P<0.001 significance probabilities are values compared with the adipocyte-derived mesenchymal stem cells; and

^{†}P<0.05, ^{††}P<0.01, and ^{†††}P<0.001 significance probabilities are values compared with the cells in Step 1; and #P<0.05 significance probabilities are values compared with the cells in Step 2;

FIGS. 3A to 3C shows drawings illustrating the experimental overview of the induced neurons and the forms thereof:

FIG. 3A: A schematic diagram illustrating the experiment on the differentiation into induced neurons from adipocyte-derived mesenchymal stem cells;

FIG. 3B: Images illustrating that induced neurons have the same shape as mature neurons, Scale bar: 100 μm; and

FIG. 3C: induced neural stem cells (iNSCs), induced neural cells (iNs);

*P<0.05, **P<0.01, ***P<0.001 significance probabilities are values compared with adipocyte-derived mesenchymal stem cells; and ^{†}P<0.05, ^{††}P<0.01, and ^{†††}P<0.001 significance probabilities are values compared with induced neural stem cells (iNSCs);

FIGS. 4A to 4C shows drawings illustrating the characteristics of induced neurons derived from adipocyte-derived mesenchymal stem cells:

FIG. 4A: Scale Bar: 20 μm;

FIG. 4B: The number of induced neurons, which express molecular markers for neuronal progenitor cells, neurons, and neuroglial cells was counted in at least three different fields. The percentage represents the proportion of the number of induced neurons that express the corresponding molecular markers among the induced neurons that express DAPI corresponding to the total cell number (mean±SEM); and

FIG. 4C: Samples of electrophysiological recordings measured from induced neurons (iNs) with typical neuronal morphology are shown. Sample images and the induction of action potential by current injection are indicated, and the protocol for current injection is below the action potential trace. The bottom trace is representative spontaneous synaptic activity obtained from induced neural cells (iNs) in voltage clamp mode (holding at −60 mV) and the magnified single current is shown below the continuous trace;

FIGS. 5A to 5D shows the optimization of the cross-differentiation protocol to GABAergic neurons:

FIG. 5A: A schematic diagram illustrating the procedure of induction from adipocyte-derived mesenchymal stem cells to GABAergic neurons;

FIG. 5B: Scale bar: 20 μm;

FIG. 5C: The number of induced GABAergic neurons, which express molecular markers for medial ganglionic eminence (MGE) and neurons, was counted in at least three different fields. The percentage represents the proportion of the number of induced GABAergic neurons that express the corresponding molecular markers among the induced neurons that express DAPI corresponding to the total cell number (mean±SEM); and

FIG. 5D: Scale bar: 100 μm;

FIGS. 6A to 6E shows drawings illustrating the functional characteristics of induced GABAergic neurons:

FIG. 6A, FIG. 6B: *P<0.05, **P<0.01, ***P<0.001 significance probabilities are values compared with adipocyte-derived mesenchymal stem cells; and ^{†}P<0.05, ^{††}P<0.01, and ^{†††}P<0.001 significance probabilities are values compared with induced GABAergic neurons on day 25 of in vitro incubation;

FIG. 6C: Scale bar: 20 μm;

FIG. 6D: The number of induced GABAergic neurons which express molecular markers for GABAergic neurons was counted in at least three different fields. The percentage represents the proportion of the number of induced GABAergic neurons that express the corresponding molecular markers among the induced GABAergic neurons that express DAPI corresponding to the total cell number (mean±SEM); and

FIG. 6E: Upper left panel shows a trace of action potential firing from induced GABAergic neurons recorded in current clamp mode (upper left panel). The protocol for current injection is below the action potential trace.

The upper right panel shows that applying ramp protocol in induced GABAergic neurons elicited fast inward current followed by depolarization of the holding voltage. Voltage was gradually ramped from −100 mV to 0 mV for 1 sec. The dotted box indicates magnification of ramp current marked with an asterisk (*).

A schematic diagram of the experimental design is shown in the bottom left panel.

FIGS. 7A to 7C shows drawings illustrating the results confirmed by a microscope and quantitative RT-PCR with respect to changes in cell morphology and Schwann cell marker genes after induction of human mesenchymal stem cells into human stem cells that hypersecrete growth factors:

FIG. 7A: microscopic images in which the changes in cell morphology are observed during the process of inducing the differentiation of human bone marrow-derived mesenchymal stem cells (hMSCs) into Schwann cells;

FIG. 7B: a gel image of PCR products of Schwann cell-specific genes (GFAP, P0, S100, CNPase, and p75NTR) in differentiated hMSCs; and

FIG. 7C: a graph in which the mRNA level of each gene is quantified by a densitometry in PCR results of Schwann cell-specific genes (*P<0.05, **P<0.001 versus uhMSC. ^{†}P<0.05 versus gfMSC).

FIGS. 8A to 8B shows drawings confirming the expression of marker proteins for Schwann cells in gfMSC via immunocytochemistry (ICC) analysis:

FIG. 8A: microscopic images by immunofluorescence staining of marker proteins for Schwann cells (GFAP, P0, S100, CNPase, and p75NTR) in differentiated hMSCs; and

FIG. 8B: a graph in which the fluorescence intensity of each protein measured from the microscopic images by immunofluorescence staining (*P<0.05, **P<0.001 versus uhMSC. ^{††}P<0.001 versus gfMSC).

FIGS. 9A to 9C shows drawings confirming the occurrence of cell cycle arrest due to the progress of differentiation in gfMSC by immunofluorescence (IF) analysis:

FIG. 9A: microscopic images stained by immunofluorescence staining of p75NTR (red) and bromodeoxyuridine (BrdU) (green) in uhMSC, gfMSC, and gfMSC⁺GM;

FIG. 9B: a graph illustrating the ratio of BrdU-positive cells in the microscopic images of A above; and

FIG. 9C: a graph illustrating the ratio of p75NTR(+)/BrdU(−) cells in the microscopic images of A above (*P<0.05, **P<0.001 versus uhMSC. ^{††}P<0.001 versus gfMSC).

FIGS. 10A to 10E shows drawings illustrating the results with respect to the secretion of various growth factors from gfMSC by growth factor analysis array:

FIG. 10A: results of microarray analysis of growth factors in media, uhMSC-CdM, and gfMSC-CdM;

FIG. 10B: a graph illustrating relative expression levels of each growth factor relative to positive control in the above results of A;

FIG. 10C: a table comparing the relative expression levels of each growth factor with those detected in media in the above results of A (*P<0.05, **P<0.001 versus media alone. ^{†}P<0.05, ^{††}P<0.001 versus uhMSC-CdM);

FIG. 10D: a graph illustrating the results of HGF expression in uhMSC, gfMSC, and gfMSC+GM confirmed by ELISA; and

FIG. 10E: a graph illustrating the results of VEGF expression in uhMSC, gfMSC, and gfMSC+GM confirmed by ELISA (*P<0.05, **P<0.001 versus CdM).

FIGS. 11F to 11J shows drawing illustrating the results of various growth factor secretion from gfMSC by growth factor analysis array:

FIG. 11F: a graph illustrating the results of HGF production by gfMSC in induced differentiation medium (SCIM) and common growth medium (GM) confirmed by ELISA;

FIG. 11G: a graph illustrating the results of VEGF production by gfMSC in induced differentiation medium (SCIM) and common growth medium (GM) confirmed by ELISA;

FIG. 11H: a gel image in which the expressions of HGF and VEGF in uhMSC, gfMSC, gfMSC+SCIM, and gfMSC+GM are confirmed by quantitative RT-PCR;

FIG. 11I: a graph in which the expression level of HGF is calibrated to a relative level with respect to GAPDH in the above results of H; and

FIG. 11J: a graph in which the expression level of VEGF is calibrated to a relative level with respect to GAPDH in the above results of H (*P<0.05, **P<0.001 versus uhMSC).

FIGS. 12A to 12F shows drawings illustrating the results of promotion of the growth and proliferation of neurites of Neuro2A cells co-cultured with gfMSC, confirmed by quantitative analysis and trypan blue staining of neurites:

FIG. 12A: a schematic diagram in which Neuro2A cells and human mesenchymal stem cells are co-cultured;

FIG. 12B: microscopic images in which the neurites of Neuro2A cells co-cultured with gfMSC were observed;

FIG. 12C: a graph illustrating the ratio of the cells having neurites with a length greater than the diameter of at least one cell body in Neuro2A cells in the above results of B;

FIG. 12D: a graph illustrating the sum of the length of neurites per cell in Neuro2A cells in the above results of B (*P<0.05, **P<0.001 versus Media. ^{†}P<0.05, ^{††}P<0.001 versus uhMSC. ^{#}P<0.05, ^{##}P<0.001 versus gfMSC);

FIG. 12E: a graph illustrating the average number of Neuro2A cells in each group after staining the Neuro2A cells, which were co-cultured with gfMSC, with trypan blue; and

FIG. 12F: a graph illustrating the ratio of apoptosized Neuro2A cells in each group after staining the Neuro2A cells, which were co-cultured with gfMSC, with trypan blue (*P<0.05, **P<0.001 versus Media. ^{†}P<0.05, ^{††}P<0.001 versus uhMSC. ^{#}P<0.05, ^{##}P<0.001 versus gfMSC).

FIGS. 13A to 13D shows drawings illustrating the results that the gfMSC transplantation increases growth of spinal nerves and cell survival in a damaged spinal cord tissue sections model confirmed by immunohistochemistry (IHC) and TUNEL analysis:

FIG. 13A: microscopic images in which the results of gfMSC transplantation on the growth of spinal nerves in a damaged spinal cord tissue sections model are confirmed by immunohistochemistry (IHC);

FIG. 13B: a graph in which the integrated optical density was normalized by the spinal cord tissue sections in the control group in the above results of A (*P<0.05, **P<0.001 versus Control. ^{††}P<0.001 versus lysolecithin-treated slices (LPC). ^{##}P<0.001 versus LPC+uhMSC. ^{§§} P<0.001 versus LPC+gfMSC);

FIG. 13C: microscopic images in which gfMSC transplantation increases the cell survival in a damaged spinal cord tissue sections model confirmed by TUNEL analysis; and

FIG. 13D: a graph in which the TUNEL-positive cells are subjected to quantitative analysis in the results of C above (**P<0.001, versus Control. ^{††}P<0.001 versus LPC. ^{#}P<0.05, ^{##}P<0.001 versus LPC+uhMSC).

FIGS. 14A to 14D shows drawings illustrating the results that the effects of exogenous HGF and VEGF as neurotrophic factors in the Neuro2A cells and a damaged spinal cord tissue sections model are confirmed through quantitative analysis, trypan blue staining, and immunohistochemistry (IHC) of neurites:

FIG. 14A: a graph illustrating the ratio of the cells having neurites with a length greater than the diameter of at least one cell body in Neuro2A cells which were cultured by adding recombinant HGF thereto;

FIG. 14B: a graph illustrating the sum of the length of neurites per cell in Neuro2A cells which were cultured by adding recombinant HGF thereto;

FIG. 14C: a graph illustrating the ratio of the cells having neurites with a length greater than the diameter of at least one cell body in Neuro2A cells which were cultured by adding recombinant VEGF thereto; and

FIG. 14D: a graph illustrating the sum of the length of neurites per cell in Neuro2A cells which were cultured by adding recombinant VEGF thereto (*P<0.05, **P<0.001 versus untreated Neuro2A cells. ^{†}P<0.05, ^{††}P<0.001 versus Neuro2A cells co-treated with HGF and VEGF).

FIGS. 15E to 15G shows drawings illustrating the effects of exogenous HGF and VEGF as neurotrophic factors in the Neuro2A cells and a damaged spinal cord tissue sections model, confirmed through quantitative analysis, trypan blue staining, and immunohistochemistry (IHC) of neurites:

FIG. 15E: microscopic images confirming that exogenous HGF and/or VEGF protein increase the growth of neurites in the damaged spinal cord tissue sections;

FIG. 15F: a graph in which the relative integrated optical density (IOD) values of nerve fibers that were NF-M immunofluorescence stained according to the concentration of HGF and/or VEGF protein were normalized to spinal cord tissue sections in the control group in the above results of E (*P<0.05, **P<0.001 versus control. ^{†}P<0.05, ^{††}P<0.001 versus spinal cord tissue treated with lysolecithin (LPC)); and

FIG. 15G: a drawing illustrating a mechanism in which human stem cells differentiated from human bone marrow-derived mesenchymal stem cells that hypersecrete a growth factor function as a neurotrophic factor by secreting HGF and VEGF in a damaged spinal nerve.

### DETAILED DESCRIPTION OF THE PREFERRED EMBODIMENTS

Hereinafter embodiments of the present invention will be described in detail with reference to accompanying drawings.

The present invention relates to a cell composition which includes neural stem cells, neurons, or GABAergic neurons differentiated from mesenchymal stem cells. More specifically, the present invention relates to a cell composition which includes neural stem cells, neurons, or GABAergic neurons differentiated from mesenchymal stem cells, which, by including neural stem cells; and at least one between immature neural stem cells and mesenchymal stem cells, can improve growth, survival rate, differentiation rate, etc. of cells at the injected site in vivo; can be applied to neurological disorders without the need to remove the cells which have not been converted to the final neural stem cells, neurons, or GABAergic neurons; can effectively prevent, improve, or treat neurological disorders; can be used as a safe cell therapeutic because the cell composition has a low probability of causing cytotoxicity and being developed into cancer cells when applied in vivo; can be utilized as an excellent source for neural stem cells, neurons, or GABAergic neurons; and can be utilized as a cosmetic composition as well as a therapeutic agent for neurological disorders.

The present invention relates to a composition for cell regeneration, which, by including at least one among neural stem cells, neurons, and GABAergic neurons; and cells that hypersecrete a growth factor, can promote cell growth in vivo or in vitro, can exhibit effects of increasing the growth of damaged neurons, nerve fibers, etc., and inhibiting cell death, can be used as a therapeutic agent for neurological disorders, even if cells at the intermediate step of conversion are included therein, can prevent, improve, or treat various neurological disorders, ischemic diseases, etc., has a low probability of causing side effects when embryonic stem cells or induced pluripotent stem cells are applied in vivo, can be utilized as a cosmetic composition, and has excellent bioadaptability.

Hereinafter, the present invention will be described in detail.

As used herein, the term “stem cell” refers to a cell which has the ability to differentiate itself into two or more cells while having a self-replication ability, and the term “adult stem cell” refers to a stem cell which appears during a stage where each organ of an embryo is formed as the developmental process progresses or during an adult stage.

As used herein, the term “prevention” refers to suppressing the expression of an epileptic seizure by administration when the prognosis of an epileptic seizure is detected; the term “treatment” refers to preventing the recurrence of an epileptic seizure; and the term “improvement of symptoms” refers to reducing or decreasing the frequency, intensity, etc. of epileptic seizures.

As used herein, the term “neurological disorder” is defined as a disorder in the nervous system and includes disorders associated with the central nervous system (brain, brainstem, and cerebellum), the peripheral nervous system (including the cerebral neurons), and the autonomic nervous system (some of which are located in both the central and peripheral nervous system). In particular, the neurological disorder includes any disease in which the function of neural crest cells or Schwann cells is impaired, altered, or impeded. Examples of neurological disorders associated with Schwann cells may include, but are not limited to, demyelinating disease, multiple sclerosis, spinal cord disorder, experimental allergic encephalomyelitis (EAE), acute diffuse encephalomyelitis (ADEM), post-infection or post-vaccination encephalomyelitis, peripheral neuropathy, Schwannoma, Charcot Marie tooth disease, Guillain-Barré Syndrome, chronic inflammatory demyelinating polyneuropathy (CIDP), etc.

The cell composition of the present invention includes neural stem cells; and at least one selected from the group consisting of immature neural stem cells and mesenchymal stem cells; wherein the number of the neural stem cells is at least 70% of the number of the cells included in the cell composition, and the total number of the cells of the immature neural stem cells and the mesenchymal stem cells is at most 30% of the number of the cells included in the cell composition.

The cell composition of the present invention may include neural stem cells greater than or equal to 70% among 100% of the cells included in the cell composition, and preferably greater than or equal to 85%, but is not limited thereto. For example, the cell composition may include 75%, 80%, 85%, 90%, 95%, 99%, etc.

The present invention provides a neural stem cell differentiated from a mesenchymal stem cell.

The composition for cell regeneration of the present invention includes at least one selected from the group consisting of neural stem cells, neurons, and GABAergic neurons; and cells that hypersecrete a growth factor.

The neural stem cell refers to a cell which maintains the potency capable of producing neurons and neuroglial cells. The neural stem cells may undergo a symmetric division and/or asymmetric division. When a neural stem cell undergoes a symmetric division, a neural stem cell can be divided to form two daughter neural stem cells or two committed progenitor cells, whereas a neural stem cell undergoes an asymmetric division, a neural stem cell can be divided to form a daughter neural stem cell and a committed progenitor cell (e.g., neural progenitor cell or neuroglial progenitor cell). The neural stem cell, which is a novel neural stem cell different from the known neural stem cells, are more excellent in required abilities with respect to cell survival and differentiation (including expression markers, proliferation rate, differentiation rate, adhesion rate, survival rate, environmental adaptability, etc.) compared to neural stem cells in the body or neural stem cells in the control group. In addition, the neural stem cell can perform one or more cell divisions, preferably 10 to 30 times, more preferably 40 or more, even more preferably 50 or more, and most preferably an unlimited number of cell divisions. The neural stem cell can undergo a symmetric division or asymmetric division, and preferably a symmetric division.

The neural stem cell can express the following neuron markers, and may express ABCG2, ASCL1/Mash1, beta-Catenin, BMI-1, Brg1, N-Cadherin, Calcitonin R, CD15/Lewis X, CD133, CDCP1, COUP-TF I/NR2F1, CXCR4, FABP7/BFABP, FABP8/M-FABP, FGF R2, FGF R4, FoxD3, Frizzled-9, GATA-2, GCNF/NR6A1, GFAP, Glut1, HOXB1, ID2, Meteorin, MSX1, Musashi-1, Musashi-2, Nestin, NeuroD1, Noggin, Notch-1, Notch-2, Nrf2, Nucleostemin, Numb, Otx2, Pax3, Pax6, PDGF R alpha, PKC zeta, Prominin 2, ROR2, RUNX1/CBFA2, RXR alpha/NR2B1, sFRP2, SLAIN1, SOX1, SOX2, SOX9, SOX11, SOX21, SSEA-1, SSEA-4, TRAF-4, ZIC1, Nestin, FoxG1, Nkx2.1, Gli3, Vimentin, Tuj1, Emx1, Olig2, Ascl1, etc., for example, express at least one among Nestin, Sox2, Sox1, Musashi-1, FoxG1, Nkx2.1, Pax6, Gli3, Vimentin, Tuj1, Emx1, Olig2, and Ascl1, and preferably, may express all of Nestin, Sox2, Sox1, Musashi-1, FoxG1, Nkx2.1, Pax6, Gli3, Vimentin, Tuj1, Emx1, Olig2, and Ascl1. According to an embodiment of the present invention, the neural stem cell may hypersecrete at least one of the markers described above, compared to neural stem cells in vivo.

The immature neural stem cells may express markers for adipose tissue-derived mesenchymal stem cells and/or neural stem cells, but may not be limited thereto. Examples of the markers for adipose tissue-derived mesenchymal stem cells may include ALCAM/CD166, aminopeptidase inhibitors, aminopeptidase N/CD13, CD9, CD44, CD90/Thy1, endoglin/CD105, ICAM-1/CD54, integrin alpha 4/CD49d, integrin alpha 4 beta 1, integrin alpha 4 beta 7/LPAM-1, integrin alpha 5/CD49e, integrin beta 1/CD29, MCAM/CD146, osteopontin/OPN, PUM2, SPARC, VCAM-1/CD106, etc., but are not limited thereto. The markers for neural stem cells may be the above-described markers, but are not limited thereto. The immature neural stem cells can improve growth, survival rate, and differentiation rate of the cells included in the cells of the composition of the present invention or the cells at the injected site in vivo. The cell composition of the present invention, by including the immature neural stem cells, can induce the effects of growth promotion, improvement of survival rate, and improvement of differentiation rate of neural stem cells, and can exhibit a more excellent therapeutic effect by the above effects when the cell composition is applied to neurological disorders.

The cell composition of the present invention may further include at least one between neurons and immature neurons.

Additionally, the present invention provides a neuron differentiated from a mesenchymal stem cell.

A neuron, which is a major cell constituting the nervous system, is a cell that can transmit a signal by an electrical method unlike other cells by expressing an ion channel (e.g., sodium channel, potassium channel, etc.). Additionally, a neuron performs functions of receiving and storing various information by transmitting and receiving signals through adjacent neurons and synapses. In other words, neurons are the cells in the nervous system and can be used interchangeably with a nerve cell or a neuronal cell. The neuron is a novel neuron which is different from the neurons in the body, and is more excellent in required abilities with respect to cell survival and differentiation (including expression markers, proliferation rate, differentiation rate, adhesion rate, survival rate, environmental adaptability, etc.) compared to neurons in the body or neurons in the control group. Additionally, since the neuron is differentiated from a mesenchymal stem cell, which is an adult stem cell, there is little concern about side effects when applied in vivo, and due to the excellent properties, and the neuron is effective in the treatment of neurological disorders (e.g., brain tumors, peripheral neuropathy, Ellis syndrome, Parkinson's disease, Alzheimer's disease, various paralysis, etc.).

The neuron can express the above-described neural stem cell markers, and additionally, can express markers such as NSE, NeuN, doublecortin (DCX), c-fos, choline acetyltransferase (ChAT), tyrosine hydrdoxylase (TH), Tau, Calbindin-D28k, calretinin, NFP, NeuroD, PSA-NCAM, Tuj1, MAP2, FoxG1, Nkx2.1, Dlx2, Dlx5, Lhx6, GAD67, SCN5A, Olig2, S100, etc., for example, at least one among Tuj1, MAP2, FoxG1, Nkx2.1, Pax6, Dlx2, Dlx5, Lhx6, GAD67, SCN5A, Olig2, S100, and NeuN, and may express all of Tuj1, MAP2, FoxG1, Nkx2.1, Pax6, Dlx2, Dlx5, Lhx6, GAD67, SCN5A, GFAP, 5100, Olig2, and NeuN. According to an embodiment of the present invention, the neuron may hypersecrete at least one of the markers described above, compared to neurons in vivo.

The cell composition of the present invention is a cell composition, which includes GABAergic neurons; and at least one selected from the group consisting of immature GABAergic neurons and mesenchymal stem cells, and the total number of the cells of the immature GABAergic neurons and the mesenchymal stem cells is at most 30% of the number of the cells included in the cell composition.

The cell composition of the present invention may further include at least one between GABAergic neurons and immature GABAergic neurons. The GABAergic neurons are neurons which produce gamma aminobutyl acid (GABA), i.e., a major inhibitory neurotransmitter of the central nervous system, and can serve as an excellent source for cell therapy for cerebral nervous system diseases (e.g., stroke, traumatic brain injury, cerebral palsy, epilepsy, Huntington's disease, etc.). The GABAergic neurons are novel cells. Although the GABAergic neurons are functionally the same as GABAergic neurons in the body in that the GABAergic neurons show a spontaneous inhibitory post-synaptic current (IPSC) when treated with a glutamic acid receptor blocker, and the IPSC disappears when treated with a GABA receptor blocker, GABAergic neurons are different from the GABAergic neurons in the body or the cells in the control group, with respect to the differentiation potency, survival rate, settlement ability, expression frequency of IPSC, expression markers, etc.

Additionally, the present invention provides a GABAergic neuron differentiated from a mesenchymal stem cell.

The cell composition of the present invention, by further including GABAergic neurons, can prevent, improve, or treat epilepsy, bipolar disorder, schizophrenia, anxiety disorders, etc. and preferably can prevent, improve, or treat epilepsy. Epilepsy refers to a chronic disease of the cerebrum characterized by paroxysmal brain dysfunction caused by excessive discharge of neurons. Examples of the symptoms of epilepsy may include epilepsy seizure, etc. The epilepsy seizure may be divided into partial (focal, local) seizures and generalized seizures. The partial seizures may include, for example, simple partial seizures, complex partial seizures (e.g., automatism, etc.), partial seizures evolving to secondarily generalized seizures, etc. The generalized seizures may include, for example, absence seizures, atypical absence seizures, myoclonic seizures, clonic seizures, tonic seizures, tonic-clonic seizures, atonic seizures (astatic), etc.

The GABAergic neurons can express markers for the above-described neurons, and additionally, can express at least one among GABA, GAT1, CALB2, GAD1, GAD2, GABA_{B} receptors 1 and 2, MAP2, Dlx2, Dlx5, GFAP, CALB2, GABRA1, GABRA2, GABRA5, GAD65, GAD67, PSD95, SYP, and NF-M, and can preferably express all of MAP2, Dlx2, Dlx5, GFAP, CALB2, GABRA1, GABRA2, GABRA5, GAD65, GAD67, PSD95, SYP, and NF-M. According to an embodiment of the present invention, the GABAergic neuron may hypersecrete at least one of the markers described above, compared to GABAergic neurons in vivo.

The immature GABAergic neurons can express markers for adipose tissue-derived mesenchymal stem cells, neural stem cells, neurons and/or GABAergic neurons, but may not be limited thereto. The markers for adipose tissue-derived mesenchymal stem cells, neural stem cells, neurons, and GABAergic neurons may be markers for the above-described adipose tissue-derived mesenchymal stem cells, neural stem cells, and GABAergic neurons, but are not limited thereto. The immature GABAergic neurons can improve growth, survival rate, and differentiation rate of the cells included in the cell composition of the present invention or the cells at the injected site in vivo. The cell composition of the present invention, by including the immature GABAergic neurons, can induce the effects of growth promotion, improvement of survival rate, and improvement of differentiation rate of GABAergic neurons, and can exhibit a more excellent effect by the above effects when the cell composition is applied to neurological disorders.

The cell that hypersecretes a growth factor may be an adult stem cell (e.g., a mesenchymal stem cell), and preferably an immature cell during the differentiation step (lineage), but is not limited thereto, and may be a Schwann-like cell or Schwann cell.

The cell that hypersecretes a growth factor may express growth factors, such as hepatocyte growth factor (HGF), vascular endothelial growth factor (VEGF), insulin-like growth factor binding protein-1 (IGFBP-1), IGFBP-2, IGFBP-4, IGFBP-6, stem cell factor (SCF), etc., and preferably VEGF and HGF, but may not be limited thereto.

The cell that hypersecretes a growth factor may express at least one marker selected from the group consisting of GFAP, P0, S100β, CNPase, and p75NTR, and may preferably express all of these markers, but may not be limited thereto.

The cell that hypersecretes a growth factor may secrete 10 times more HGF and/or VEGF than bone marrow-derived mesenchymal stem cells, but may not be limited thereto.

The cell that hypersecretes a growth factor can increase the growth of neurites and growth and survival of cells, and thus the cell, by being included in the composition for cell regeneration of the present invention, can increase the growth and survival rate of neural stem cells, neurons, and GABAergic neurons, and accordingly, can exhibit a very excellent effect with regard to the treatment of neurological disorders.

All the cells included in the cell composition of the present invention are derived from mesenchymal stem cells, which are adult stem cells, and can be used as a safe cell therapeutic because the cell composition has a low probability of causing cytotoxicity when applied in vivo. When the cell composition of the present invention is used for the prevention, improvement, or treatment of neurological disorders, the number of the neural stem cells, neurons and/or GABAergic neurons to be included in the composition may be variously adjusted as necessary. For example, the composition may include the number of cells optimized for the settling rate and survival rate of the cells, and may include the number of cells that can be effective in treating a patient's disease. The cell composition of the present invention may exhibit more excellent improvement and/or therapeutic effects in the treatment of neurological disorders by including a combination of two among the neural stem cells, neurons, and GABAergic neurons. When the cell composition of the present invention is applied for treatment, the cell composition including only the cells but excluding the remaining components in the composition may be applied, but the method is not limited thereto.

According to an embodiment of the present invention, the cell that hypersecretes a growth factor may be derived from a mesenchymal stem cell.

The mesenchymal stem cells are undifferentiated stem cell isolated from the tissues of human or mammals, and may be derived from various tissues, and in particular, umbilical cord-derived mesenchymal stem cells, umbilical cord blood-derived mesenchymal stem cells, bone marrow-derived mesenchymal stem cells, adipose-derived mesenchymal stem cells, muscle-derived mesenchymal stem cells, neuron-derived mesenchymal stem cells, skin-derived mesenchymal stem cells, amnion-derived mesenchymal stem cells, and placenta-derived mesenchymal stem cells, and the techniques for isolating stem cells from each tissue are known in the art. More preferably, the mesenchymal stem cells may be obtained from humans, pigs, cows, sheep, rabbits, mice, or rats; more preferably humans, pigs, or mice; and more preferably bone marrow, umbilical cord blood, or adipose tissue of humans. For example, mesenchymal stem cells may be adipose tissue-derived cells or bone marrow-derived cells.

According to an embodiment of the present invention, the mesenchymal stem cell may be an adipose-derived or bone marrow-derived mesenchymal stem cell, and preferably the neural stem cell, neuron, and GABAergic neuron may be differentiated from an adipose-derived mesenchymal stem cell, and the cell hypersecreting a growth factor may be differentiated from a bone marrow-derived mesenchymal stem cell, but is not limited thereto.

The mesenchymal stem cells may be obtained by the methods described below. For example, the adipose tissues are cut and the cells are outgrown by directly cultivation of the cut adipose tissues. Then, the grown cells are subcultured so as to obtain mesenchymal stem cells. As the medium used in the above cultivation, any medium conventionally used in animal cell culture, for example, Eagles' MEM, α-MEM, Iscove's MEM, 199 medium, CMRL 1066, RPMI 1640, F12, F10, DMEM, a mixture of DMEM and F12, Way-mouth's MB752/1, McCoy's 5A, MCDB series, etc. may be used. In the subculture, the cells are cultured until 70-80% of confluency is obtained, followed by harvesting adherent cells and using a part of the harvested cells for the subsequent subculture. Through the above process, mesenchymal stem cells having differentiation potency into mesodermal tissues (e.g., osteoblasts, adipocytes, chondrocytes, etc.) and stem cell characteristics are produced.

The neural stem cell, neuron, and GABAergic neuron of the present invention may express markers for adipose tissue-derived mesenchymal stem cells, and the composition for cell regeneration may include those cells which are in the intermediate stage of differentiation from mesenchymal stem cells into neural stem cells, neurons, GABAergic neurons (i.e., undifferentiated cells). For example, the undifferentiated cells may be mesenchymal stem cells. The undifferentiated cells can express markers for the known mesenchymal stem cells, neural stem cells, neurons, GABAergic neurons. Examples of the markers for adipose tissue-derived mesenchymal stem cells may include ALCAM/CD166, aminopeptidase inhibitors, aminopeptidase N/CD13, CD9, CD44, CD90/Thy1, endoglin/CD105, ICAM-1/CD54, integrin alpha 4/CD49d, integrin alpha 4 beta 1, integrin alpha 4 beta 7/LPAM-1, integrin alpha 5/CD49e, integrin beta 1/CD29, MCAM/CD146, osteopontin/OPN, PUM2, SPARC, VCAM-1/CD106, etc., but are not limited thereto. The markers for the neural stem cells, neurons, and GABAergic neurons may be those described above, but are not limited thereto. The undifferentiated cells can improve growth, survival rate, and differentiation rate of the neural stem cells, neurons, and GABAergic neurons and the cells included in the composition for cell regeneration of the present invention and the cells at the injected site in vivo. The neural stem cells, neurons, and GABAergic neurons, and the composition for cell regeneration of the present invention, by including the undifferentiated cells, can induce the effects of growth promotion, improvement of survival rate, and improvement of differentiation rate of neural stem cells, neurons, and GABAergic neurons, and can exhibit a more excellent effect by the above effects when the cell composition and a composition for cell regeneration are applied to neurological disorders. Additionally, the undifferentiated cells can express markers which can be expressed in at least one cell selected from astrocytes and oligodendrocytes. The neural stem cells, neurons, and GABAergic neurons, and the composition for cell regeneration of the present invention may include the undifferentiated cells in a range to be at most 30% among 100% of the cells included in the composition, preferably in a range of 15% to 20%, more preferably 10%, and even more preferably 5%, but is not limited thereto, and may include for example, 1%.

The neural stem cells, neurons, and GABAergic neurons, and the cells included in the composition for cell regeneration of the present invention are all derived from mesenchymal stem cells, which are adult stem cells, and can be used as a safe cell therapeutic because they have a low probability of causing cytotoxicity when applied in vivo. For example, when neural stem cells, neurons, and GABAergic neurons, and the composition for cell regeneration are used as a cell therapeutic for the prevention, improvement, or treatment of neurological disorders, the number of these cells to be included in the composition and the cells that hypersecrete a growth factor, and the ratio of these cells (when two or more kinds of these cells are used), etc., may vary upon necessity, but may not be limited thereto. For example, the number of cells optimized for the settling rate and survival rate of the neural stem cells, neurons and/or GABAergic neurons may be included, and the number of the neural stem cells, neurons, and GABAergic neurons and the cells that hypersecrete a growth factor which are effective in treating a patient's disease may be included. The composition for cell regeneration of the present invention can exhibit a more excellent effect in prevention, improvement, or treatment of neurological disorders. When the composition for cell regeneration of the present invention is applied for treatment of neurological disorders, ischemic diseases, etc., the composition may be used after excluding the components other than the cells therein, but the method of application is not limited thereto.

The neural stem cells, neurons, and GABAergic neurons, and the composition for cell regeneration of the present invention can be applied to treatment as the composition itself without the need to remove undifferentiated cells, and thus, the cell composition is safe because the risk of occurrence of cancer, differentiation into unwanted cells, toxicity to surrounding cells, etc., which are the problems that may arise from using undifferentiated cells without removing the same from a composition for cell regeneration containing cells differentiated from embryonic stem cells or induced pluripotent stem cells, is very low. Additionally, due to the elimination of the need for removal, the effort and troubles of going through a separate removal process can be reduced, and in particular, in the case of adipose-derived mesenchymal stem cells, a large scale production is possible because the cell composition can be obtained in a large amount in vivo (e.g., abdominal fat).

The subject to which the neural stem cells, neurons, and GABAergic neurons, and the composition for cell regeneration of the present invention can be applied may be a subject which has provided the mesenchymal stem cells, and the subject may be a mammal, more preferably anthropoids (e.g., chimpanzees and gorillas), experimental animals (e.g., rats, rabbits, guinea pigs, hamsters, dogs, and cats), etc., and most preferably, anthropoids or humans, but the subject may not be limited thereto.

The neural stem cells, neurons, and GABAergic neurons, and the composition for cell regeneration of the present invention may be administered orally or parenterally, preferably in a parenteral administration mode, and most preferably, by intravenous administration and administration within brain tissue (e.g., hippocampus). The appropriate dose of the cell composition, the neural stem cells, neurons, and GABAergic neurons of the present invention may be variously prescribed by factors, such as formulation method, administration mode, age, weight, sex, condition, and diet of a patient, administration time, administration route, excretion rate, and reaction sensitivity. The number of cells to be administered, based on adults, is 10² cells to 10¹⁰ cells per day.

The neural stem cells, neurons, and GABAergic neurons, and the composition for cell regeneration of the present invention can exhibit a more excellent effect in the prevention, improvement, and treatment of neurological disorders. The term “effect” may mean to slow down the disease progression and/or terminate the disease; to have a higher effect with fewer cells than was required for conventional cell therapy; or to be able to treat, by using the cells of the present invention, a disease which could not be treated with the same cells in vivo, etc., but the effect may not be limited thereto. Additionally, the neural stem cells, neurons, and GABAergic neurons, and the composition for cell regeneration of the present invention may be applied to neurological disorders, and examples of the neurological disorders include disorders associated with the central nervous system (brain, brainstem, and cerebellum), the peripheral nervous system (including the cerebral neurons), and the autonomic nervous system (some of which are located in both the central and peripheral nervous system). In particular, the neurological disorders include any disease in which the function of neural crest cells or Schwann cells is impaired, altered, or impeded. Examples of the neurological disorders may include, but are not limited to, neurological disorders associated with Schwann cells, such as demyelinating disease, multiple sclerosis, spinal cord disorder, experimental allergic encephalomyelitis (EAE), acute diffuse encephalomyelitis (ADEM), post-infection or post-vaccination encephalomyelitis, peripheral neuropathy, Schwannoma, Charcot Marie tooth disease, Guillain-Barré Syndrome, chronic inflammatory demyelinating polyneuropathy (CIDP), etc., stroke, Parkinson's disease, Alzheimer's disease, Pick's disease, Huntington's disease, amyotrophic lateral sclerosis, traumatic central nervous system diseases, spinal cord injury disease, etc.

Examples of the neural stem cells, neurons, and GABAergic neurons, and the composition for cell regeneration of the present invention may further include B27, N2, ascorbic acid, epidermal growth factor (EGF), basic fibroblast growth factor (bFGF), purmorphamine, dibutyryl-cyclic AMP (dbcAMP), BDNF, sodium pyruvate, glutamine, insulin, transferrin, sodium selenite, or a combination thereof.

B27 and N2, which are serum-free supplements, are components of the medium used in the method of the present invention. bFGF is a protein belonging to the FGF family which functions as a cell division promoting factor (e.g., cell proliferation, cell differentiation, etc.), angiogenesis factor, bone formation factor, and nerve growth factor, and also called FGF2. bFGF is known to activate mainly receptor proteins including FGFR 1b, FGFR 1c, FGFR 2c, FGFR 3c, and FGFR 4c, and in particular, strongly activate FGFR 1c and FGFR 3c. The materials which can transmit the signal similar to that of bFGF by including an FGF family protein that activates the FGFR can be used without limitation. Insulin is a peptide hormone secreted by β cells of Langerhans islets of pancreas. Transferrin, which is a type of β globulin, is an iron transporting protein that binds to two molecules of trivalent iron ions absorbed into the serum and supplies irons for cellular proliferation or hemoglobin production mediated by transferrin receptors. Sodium selenite is an inorganic compound having the formula of Na₂SeO₃.

When the neural stem cells, neurons, and GABAergic neurons of the present invention are used as cell therapeutics, all of the materials that can be administered for cell therapy can be administered together, and may be further included to a composition for cell regeneration. Examples of such materials may include, for example, cytokine peptide factors that induce differentiation, growth, etc. of neurons and neural tissues, such as vascular endothelial growth factor (VEGF), hepatocyte growth factor (HGF), nerve growth factor (NGF), glial cell line-derived neurotrophic factor (GDNF), platelet-derived growth factor (PDGF), etc.

The neural stem cells, neurons, and GABAergic neurons, and composition for cell regeneration of the present invention may further include an extracellular matrix, which is derived from nature or an artificial production (assembled product), for example, materials (e.g., collagen, proteoglycan, fibronectin, hyaluronic acid, tenascin, entactin, elastin, fibrillin, and laminin) or fragments thereof. These extracellular matrices can be used in combination, for example, preparations from cells (e.g., BD Matrigel (trademark)), etc. may also be used.

The neural stem cells, neurons, and GABAergic neurons, and composition for cell regeneration of the present invention may further include laminin or fragments thereof. Laminin is a protein having a heterotrimeric structure having each of a chain, β chain, and γ chain, and although not particularly limited thereto, in an embodiment, the α chain is α1, α2, α3, α4, or α5; β chain is β1, β2, or β3; and γ chain is γ1, γ2, or γ3.

The neural stem cells, neurons, and GABAergic neurons, and composition for cell regeneration of the present invention may further include a neurotrophic factor. A neurotrophic factor is a ligand to a membrane receptor that plays an important role in the survival and maintenance of functioning of motor neurons. Examples of the neurotrophic factor may include nerve growth factor (NGF), brain-derived neurotrophic factor (BDNF), neurotrophin 3 (NT-3), neurotrophin 4/5 (NT-4/5), neurotrophin 6 (NT6), basic FGF, acidic FGF, FGF-5, epidermal growth factor (EGF), hepatocyte growth factor (HGF), insulin, insulin-like growth factor 1 (IGF 1), insulin-like growth factor 2 (IGF 2), glial cell line-derived neurotrophic factor (GDNF), TGF-b2, TGF-b3, interleukin 6 (IL-6), ciliary neurotrophic factor (CNTF), LIF, etc. Since the neurotrophic factor is commercially available from Wako, R & D systems, etc., the factor may be used easily, but may also be obtained by forced expression into cells according to methods known to those skilled in the art.

The neural stem cells, neurons, and GABAergic neurons, and composition for cell regeneration of the present invention may be cultured in a medium. When these cells are cultured in a medium, the culture (especially, the supernatant) may be utilized as a cosmetic composition, a therapeutic for the prevention, improvement, or treatment of neurological disorders, etc., but may not be limited thereto.

The neural stem cells, neurons, and GABAergic neurons, and composition for cell regeneration of the present invention may be prepared in a unit dosage form by formulation using a pharmaceutically acceptable carrier and/or excipient, or incorporated into a multi-dose container. In particular, the formulations may be in the form of solutions, suspending agents, syrups, or emulsions; or in the form of extracts, powders, granules, tablets or capsules, and a dispersant or stabilizer may be further included. The carrier is commonly used in preparation, and examples of the carrier may include lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, saline, phosphate buffered saline (PBS), media, etc., but the carrier is not limited thereto.

Examples of the suspending agents may include methyl cellulose, polysorbate 80, hydroxyethyl cellulose, gum arabic, gum tragacanth, sodium carboxymethyl cellulose, polyoxyethylene sorbitan monolaurate, etc. Examples of the solution adjuvant may include polyoxyethylene hydrogenated castor oil, polysorbate 80, nicotinic acid amide, polyoxyethylene sorbitan monolaurate, Macrogol, castor oil fatty acid ethyl ester, etc.; examples of the stabilizer may include dextran 40, methyl cellulose, gelatin, sodium sulfite, sodium metasulfate, etc.; and examples of the isotonic agent may include D-mannitol and sorbitol. Examples of the preservatives may include methyl paraoxybenzoate, ethyl paraoxybenzoate, sorbic acid, phenol, cresol, chlorocresol, etc.;

examples of the adsorption inhibitor may include human serum albumin, lecithin, dextran, an ethylene oxide-propylene oxide copolymer, hydroxypropyl cellulose, methyl cellulose, polyoxyethylene hydrogenated castor oil, polyethylene glycol, etc.; and

examples of the sulfur-containing reducing agent may include those having a sulfhydryl group (e.g., N-acetylcysteine, N-acetyl homocysteine, thioctoic acid, thiodiglycol, thioethanolamine, thioglycerol, thiosorbitol, thioglycolic acid and salts thereof, sodium thiosulfate, glutathione, thioalkanoic acid having 1 to 7 carbon atoms, etc.); and examples of the antioxidant may include chelating agents (e.g., erythorbic acid, dibutylhydroxytoluene, butylhydroxyanisole, α-tocopherol, tocopherol acetate, L-ascorbic acid and salts thereof, L-ascorbic acid palmitate, L-ascorbic acid stearate, sodium hydrogen sulfite, sodium sulfite, triamyl gallate, propyl gallate or sodium ethylenediaminetetraacetate (EDTA), sodium pyrophosphate, sodium metaphosphate, etc.). Examples of the lyophilizing agent may include DMSO, glycerol, etc., but is not limited thereto, and furthermore, may include commonly added components such as inorganic salts (e.g., sodium chloride, potassium chloride, calcium chloride, sodium phosphate, potassium phosphate, sodium hydrogen carbonate, etc.) and organic salts (e.g., sodium citrate, potassium citrate, sodium acetate, etc.).

The neural stem cells, neurons, GABAergic neurons and the composition for cell regeneration may include neural stem cells, neurons, and GABAergic neurons differentiated from the mesenchymal stem cell and the cells that hypersecrete a growth factor, obtained from the subject, to which these cells are to be administered, but may not be limited thereto.

The neural stem cells, neurons, GABAergic neurons and the composition for cell regeneration of the present invention may be applied to ischemic diseases, for example, ischemic diseases associated with trauma, rejections at transplantation, ischemic cerebrovascular disorder, ischemic kidney disease, ischemic lung disease, infectious disease, and ischemic disease of the extremities and ischemic heart disease, etc., but may not be limited thereto.

The neural stem cells, neurons, GABAergic neurons and the composition for cell regeneration of the present invention may be a medium.

When the neural stem cells, neurons, GABAergic neurons, and the composition for cell regeneration of the present invention are media, the media may be prepared using a medium for animal cell culture as a base medium. Examples of the base medium may include Glasgow's Minimal Essential Medium (GMEM) medium, IMDM medium, Medium 199 medium, Eagle's Minimum Essential Medium (EMEM) medium, αMEM medium, Dulbecco's modified Eagle's Medium (DMEM) medium, Ham's F12 medium, RPMI 1640 medium, Fischer's medium, Neurobasal Medium (Life Technologies), a mixed medium thereof, etc. The medium may contain serum or the medium may be serum-free. Upon necessity, the medium may one or more serum replacements (e.g., albumin, transferrin, knockout serum replacement (KSR)(serum replacement of FBS at the time of culturing ES cells), N2 supplement (Invitrogen), B27 supplement (Invitrogen), fatty acids, insulin, collagen precursors, trace elements, 2-mercaptoethanol, 3′-thiolcglycerol, etc.), and may include one or more materials selected from fats, amino acids, L-glutamine, glutamax (Invitrogen), non-essential amino acids, vitamins, proliferation factors, low molecular weight compounds, antibiotics, antioxidants, pyruvate, buffers, inorganic salts, nucleic acids (e.g., dibutyryl-cyclic AMP (dbcAMP), etc.). Additionally, the cell composition for cell regeneration of the present invention may further include fetal calf serum (FCS), fetal bovine serum (FBS), etc. as well as antibiotics, growth factors, amino acids, inhibitors, or analogs thereof. As such materials, lipoic acid, albumin, hydrocortisone, insulin, etc. may be further included, but the materials are not limited thereto. Additionally, a neurotrophic factor may be added appropriately. The culture fluid obtained from the above medium may be used for the prevention, improvement, or treatment of neurological disorders, and may be used as a cosmetic composition.

Hereinafter, the present invention will be described in detail by Examples and Experimental Examples. However, these Examples and Experimental Examples are provided only for illustrating purposes, and the present invention is not limited by the following Examples and Experimental Examples.

### EXAMPLES

Cultivation of Adipocyte-Derived Mesenchymal Stem Cells (hADSCs)

In order to maintain adipocyte-derived mesenchymal stem cells, the cells were cultured in a medium containing 10% fetal bovine serum (FBS) and 1% penicillin/streptomycin to DMEM base medium, and maintained in an incubator (37° C., 5% CO₂). Then, cells were removed by treating with 0.25% trypsin-EDTA in a cultured dish where the cells were cultured, added into each of a 10 cm² tissue culture dish with a density of 8×10⁴ cells/dish, and then subcultured at 4-day intervals. Each medium was replaced with a fresh medium every other day and the passage of subculture of cells were controlled not to exceed 9 subcultures.

### <Experimental Example 1> Confirmation of Establishment of Cross-Differentiation Method into Neural Stem Cell-Like Cells Derived from Adipose Tissue-Derived Stem Cells

<1-1> Establishment of Three-Step Differentiation Conditions

For the method of cross-differentiation of adipocyte-derived mesenchymal stem cells into neural stem cells, an initial comparison experiment was performed by dividing conditions into a condition in which 3% knock out serum replacement (KOSR) was added with no treatment and a condition in which 3% KOSR was added along with the treatment with a small molecule inhibitor, so as to establish the differentiation conditions.

Differentiation is largely divided into three steps, and in a first step, 2×10⁵ adipocyte-derived mesenchymal stem cells were added into a gelatin-coated dish (6 cm²) and cultured in an incubator (37° C., 5% CO₂) for about one day. On the next day, the medium was replaced with a pre-induction medium. The medium composition contained 3% KOSR, 1% penicillin/streptomycin, 1% glutamax, 3 mM D-glucose, 1% non-essential amino acids, and 4 ng/mL basic fibroblast growth factor (bFGF) to the base medium (DMEM F-12), and the stem cells were maintained in an incubator (37° C., 5% CO₂) for 8 days. In this step, the conditions were divided into two groups: one group treated with 10 μM SB431542, 0.1 μg/mL Noggin, and 0.5 μM LDN193289 (i.e., small molecule inhibitors); and the other group not treated with the same, and the differentiation into neural stem cells were compared.

Then, in a second step, the medium composition was replaced with a neural induction medium. The neural induction medium contained 1% glutamax, 3 mM D-glucose, 0.2 mM ascorbic acid, 1 mM sodium pyruvate, 2% B27, and 1% N2 to the base medium (DMEMF-12:neurobasal (1:1)), and incubated in an incubator (37° C., 5% CO₂) for 5 days.

Finally, in a third step, to increase the number of neural stem cell-like cells, the medium was replaced with a growth medium, in which bFGF (20 ng/mL) and epidermal growth factor (EGF; 20 ng/mL) were contained to the neural induction medium, and incubated in an incubator (37° C., 5% CO₂) for 7 days. Then, in order to confirm whether the neural stem cells were appropriately differentiated, the cells obtained after completing these three-steps were washed with PBS, treated with 1× TryPLE select in an incubator at 37° C. for 3 to 4 minutes, and well ground into single cells. In addition, the cells were attached to a dish (6 cm²), which was coated in advance with poly-L-ornithine (PLO; 10 μg/mL)/fibronectin (FN; 1.0 μg/mL), and to a 4-well dish, in which coverslips (12 mm²) were added after coating with PLO (50 μg/mL)/fibronectin (FN; 5 μg/mL), and cultured in an incubator (37° C., 5% CO₂) for 2 to 3 days, followed by analyzing the differentiation of cells. The dish (6 cm²) was subjected to real-time PCR analysis to compare the expression of target genes, and the 4-well dish having the coverslips was subjected to observation of protein expression and the form of expression via immunofluorescence staining analysis.

<1-2> Confirmation of Gene Expression Levels Associated with Differentiation of Induced Neural Stem Cells Via Real-Time PCR

To analyze the gene expression level of the differentiation-induced neural stem cells, sampling was performed using a cell scraper and total RNA was isolated using a Trizol reagent according to the manual method. The isolated total RNA was synthesized into cDNA by M-MLV reverse transcriptase at 42° C. for 1 hour. Gene expression was analyzed via SYBR Green gene expression assay using the synthesized CDNA template, and the target genes and primer sequences to be compared are shown in Table 1 below. The target gene expression level was normalized by endogenous GAPDH, and the comparison of gene levels was performed by the Ct comparative method of the measured genes. The Ct value is the cycle number at which the fluorescence level reached threshold, and the ΔCt value was determined by subtracting the Ct value of the GAPDH value from the Ct value of the target gene [ΔCt=Ct (target)−Ct (GAPDH)]. The standard expression gene was indicated as the relative value ΔCt of target genes of GAPDH=2^{ΔCt}.

TABLE 1

| Name of | | Product | Gene Bank |
|---|---|---|---|
| Gene | Forward (F) & reverse (R) primer sequences | size (bp) | Accession |
| CALB2 | F: 5′-CTCCAGGAATACACCCAAA-3′ (SEQ ID No: 1) | 207 | BC015484.2 |
| | R: 5′-CCAGCTCATGCTCGTCAATGT-3′ (SEQ ID No: 2) | | |
| Dlx2 | F: 5′-GCACATGGGTTCCTACCAGT-3′ (SEQ ID No: 3) | 153 | BC032558.1 |
| | R: 5′-TCCTTCTCAGGCTCGTTGTT-3′ (SEQ ID No: 4) | | |
| Dlx5 | F: 5′-CCAACCAGCCAGAGAAAGAA-3′ (SEQ ID No: 5) | 150 | BC006226 |
| | R: 5′-GCAAGGCGAGGTACTGAGTC-3′ (SEQ ID No: 6) | | |
| Emx1 | F: 5′-AAGCGCGGCTTTACCATAGAG-3′ (SEQ ID No: 7) | 150 | NM_004097.2 |
| | R: 5′-GCTGGGGTGAGGGTAGTTG-3′ (SEQ ID No: 8) | | |
| FoxG1 | F: 5′-AGAAGAACGGCAAGTACGAGA-3′ (SEQ ID No: 9) | 189 | BC050072.1 |
| | R: 5′-TGTTGAGGGACAGATTGTGGC-3′ (SEQ ID No: 10) | | |
| GABRA1 | F: 5′-GGATTGGGAGAGCGTGTAACC-3′ (SEQ ID No: 11) | 66 | BC030696 |
| | R: 5′-TGAAACGGGTCCGAAACTG-3′ (SEQ ID No: 12) | | |
| GABRA2 | F: 5′-GTTCAAGCTGAATGCCCAAT-3′ (SEQ ID No: 13) | 160 | BC022488 |
| | R: 5′-ACCTAGAGCCATCAGGAGCA-3′ (SEQ ID No: 14) | | |
| GABRA5 | F: 5′-ATCTTGGATGGGCTCTTGG-3′ (SEQ ID No: 15) | 130 | BC111979 |
| | R: 5′-TGTACTCCATTTCCGTGTCG-3′ (SEQ ID No: 16) | | |
| GAD65 | F: 5′-GGTGGCTCCAGTGATTAAAG-3′ (SEQ ID No: 17) | 165 | M81882.1 |
| | R: 5′-TGTCCAAGGCGTTCTATTTC-3′ (SEQ ID No: 18) | | |
| GAD67 | F: 5′-AGGCAATCCTCCAAGAACC-3′ (SEQ ID No: 19) | 218 | M81883.1 |
| | R: 5′-TGAAAGTCCAGCACCTTGG-3′ (SEQ ID No: 20) | | |
| GFAP | F: 5′-CAACCTGCAGATTCGAGAAA-3′ (SEQ ID No: 21) | 153 | AF419299.1 |
| | R: 5′-GTCCTGCCTCACATCACATC-3′ (SEQ ID No: 22) | | |
| Gli3 | F: 5′-TGGTTACATGGAGCCCCACTA-3′ (SEQ ID No: 23) | 116 | M57609.1 |
| | R: 5′-GAATCGGAGATGGATCGTAATGG-3′ (SEQ ID No: 24) | | |
| LHX6 | F: 5′-GGGCGCGTCATAAAAAGCAC-3′ (SEQ ID No: 25) | 108 | BC103936 |
| | R: 5′-TGAACGGGGTGTAGTGGATG-3′ (SEQ ID No: 26) | | |
| Map2 | F: 5′-CGCTCAGACACCCTTCAGTAAC-3′ (SEQ ID No: 27) | 122 | U01828.1 |
| | R: 5′-AAATCATCCTCGATGGTCACAAC-3′ (SEQ ID No: 28) | | |
| Mash1 | F: 5′-TGCACTCCAATCATTCACG-3′ (SEQ ID No: 29) | 146 | NM_004316 |
| | R: 5′-GTGCGTGTTAGAGGTGATGG-3′ (SEQ ID No: 30) | | |
| Musashi | F: 5′-TTCGGGTTTGTCACGTTTGAG-3′ (SEQ ID No: 31) | 250 | AB012851.1 |
| | R: 5′-GGCCTGTATAACTCCGGCTG-3′ (SEQ ID No: 32) | | |
| Nestin | F: 5′-CACCTGTGCCAGCCTTCTTA-3′ (SEQ ID No: 33) | 170 | NM_006617 |
| | R: 5′-TTTCCTCCCACCCTGTGTCT-3′ (SEQ ID No: 34) | | |
| NKX2.1 | F: 5′-GTGAGCAAGAACATGGCCC-3′ (SEQ ID No: 35) | 182 | BC006221.2 |
| | R: 5′-AACCAGATCTTGACCTGCGT-3′ (SEQ ID No: 36) | | |
| Olig2 | F: 5′-GCTGCGACGACTATCTTCCC-3′ (SEQ ID No: 37) | 244 | NM_005806.3 |
| | R: 5′-GCCTCCTAGCTTGTCCCCA-3′ (SEQ ID No: 38) | | |
| Pax6 | F: 5′-AGGTATTACGAGACTGGCTCC-3′ (SEQ ID No: 39) | 104 | AY047583 |
| | R: 5′-TCCCGCTTATACCTGGGCTATTT-3′ (SEQ ID No: 40) | | |
| SCN5A | F: 5′-GGATCGAGACCATGTGGGAC-3′ (SEQ ID No: 41) | 151 | BC144621 |
| | R: 5′-GCTGTGAGGTTGTCTGCACT-3′ (SEQ ID No: 42) | | |
| Sox1 | F: 5′-AGATGCCACACTCGGAGATCA-3′ (SEQ ID No: 43) | 184 | NM_005986 |
| | R: 5′-GAGTACTTGTCCTCCTTGAGCAGC-3′ (SEQ ID No: 44) | | |
| Sox2 | F: 5′-AGTCTCCAAGCGACGAAAAA-3′ (SEQ ID No: 45) | 141 | NM_003106.3 |
| | R: 5′-GCAAGAAGCCTCTCCTTGAA-3′ (SEQ ID No: 46) | | |
| TuJ1 | F: 5′-GGCCTTTGGACATCTCTTCA-3′ (SEQ ID No: 47) | 241 | BC000748.2 |
| | R: 5′-ATACTCCTCACGCACCTTGC-3′ (SEQ ID No: 48) | | |
| Vimentin | F: 5′-AGAACTTTGCCGTTGAAGCTG-3′ (SEQ ID No: 49) | 255 | NM_003380.3 |
| | R: 5′-CCAGAGGGAGTGAATCCAGATTA-3′ (SEQ ID No: 50) | | |
| GAPDH | F: 5′-GTCAGTGGTGGACCTGACCT-3′ (SEQ ID No: 51) | 256 | BC083511.1 |
| | R: 5′-CACCACCCTGTTGCTGTAGC-3′ (SEQ ID No: 52) | | |

<1-3> Confirmation of Expression of Differentiation-Related Protein of Induced Neural Stem Cells by Immunofluorescence Staining

The induced neural stem cells which had been maintained in a 4-well dish were fixed using 4% formaldehyde for 15 minutes, washed twice with phosphate buffered saline (PBS) containing calcium ions and magnesium ions, and treated twice with 0.1% Triton X-100 (a surfactant) diluted in PBS, at 10 minute intervals for the permeation of antibodies.

Then, 5% goat serum diluted in 0.1% Triton X-100/PBS was added to prevent the detection caused by the adhesion of nonspecific antibodies and reacted with the sample for 1 hour. The primary antibodies varied depending on the cells to be adhered and the amount of target antibodies and dilution depending on proteins are shown in Table 2 below.

TABLE 2

| Antibody | Company | Antibody | Company |
|---|---|---|---|
| Name | (Cat. No.) | Name | (Cat. No.) |
| Anti-DLX2 | Santa Cruz | Anti-NKX2.1 | Merck Millipore |
| | (sc-81960) | | (MAB5460) |
| Anti-GABA | Sigma-Aldrich | Anti-OLIG2 | From lab stocks |
| | (A2052) | | (Gift of Harvard |
| | | | University) |
| Anti-GAD | Merck Millipore | Anti-PAX6 | Merck Millipore |
| | (AB1511) | | (MAB5554) |
| Anti-GFAP | Merck Millipore | Anti-PSD96 | Merck Millipore |
| | (MAB3402) | | (MABN68) |
| Anti-MAP2 | Merck Millipore | Anti-S100 | Dako(Z0311) |
| | (MAB3418) | | |
| | Merck Millipore | Anti-SOX2 | Merck Millipore |
| | (AB5622) | | (AB5603) |
| Anti-NCAM | BD bioscience | Anti-SYP | Sigma-Aldrich |
| | (562794) | | (SAB4502906) |
| Anti-Nestin | BD bioscience | Anti-Tuj1 | Bio Legend |
| | (611658) | | (PRB-435P) |
| Anti-NeuN | Merck Millipore | | |
| | (MAB377) | | |
| Anti-NFM | Merck Millipore | | |
| | (AB1987) | | |

Primary antibodies were attached and then allowed to react in a shaker at 4° C. for 16 hours. Secondary antibodies were selected and used depending on the host and wavelength of the primary antibodies. The goat anti-(mouse IgG)-conjugated Alexa Fluor 555 (1:200 dilution), goat anti-(mouse IgG)-conjugated Alexa Fluor 488 (1:200 dilution), goat anti-(rabbit IgG)-conjugated Alexa Fluor 555 (1:200 dilution), and goat anti-(rabbit IgG)-conjugated Alexa Fluor 488 (1:200 dilution) were used. Additionally, nuclei were stained using DAPI (1:1000 dilution).

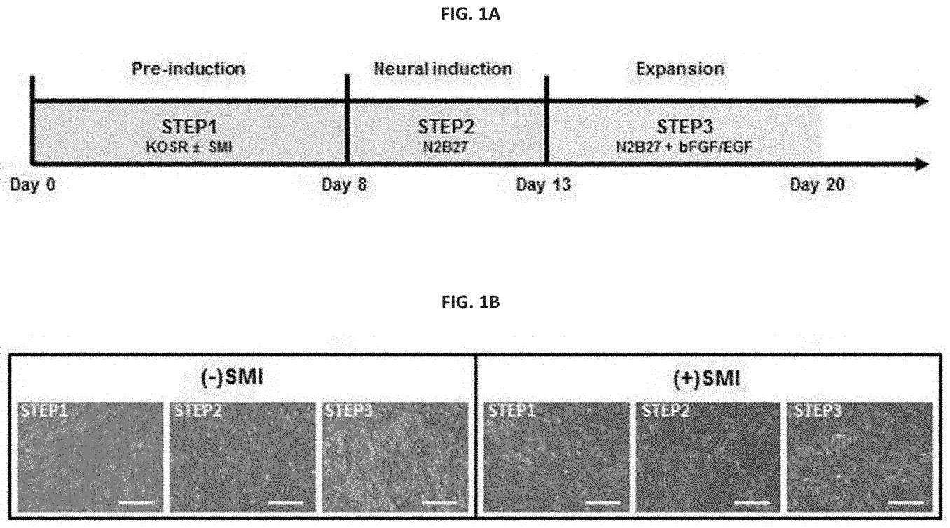

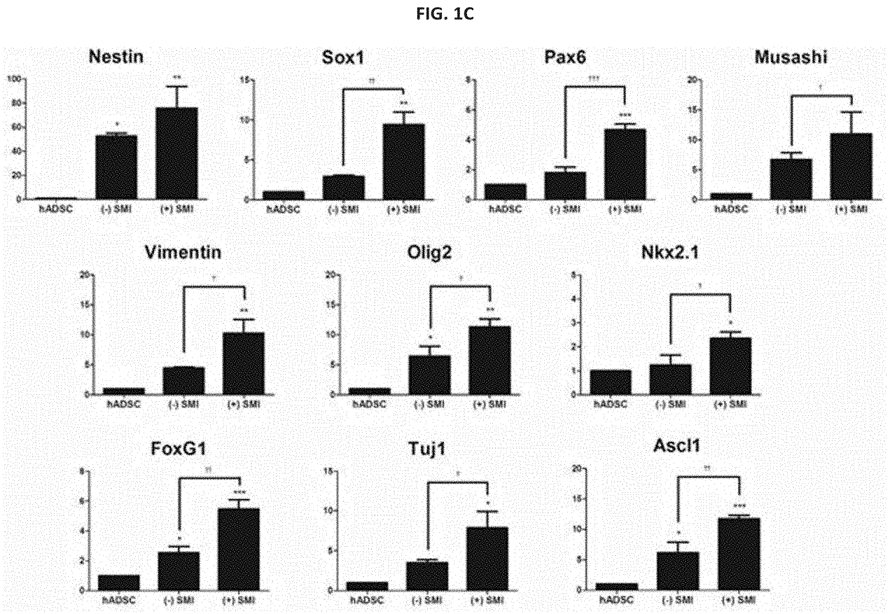

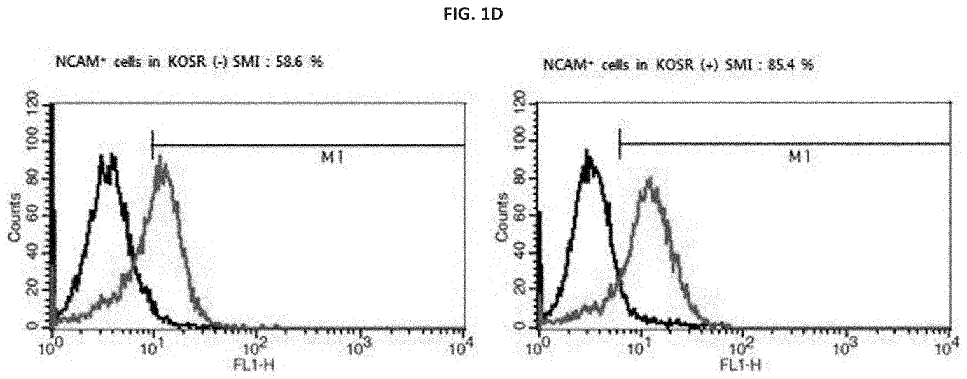

The samples which were completely stained were photographed and analyzed using Carl Zeiss LSM700 (a confocal laser-scanning microscope), and the procedure is illustrated in FIG. 1A.

As a result, as shown in FIGS. 1B to 1D, the size of the induced neural stem cells was decreased both in the group treated with a small molecule inhibitor and the group not treated with a small molecule inhibitor during the cross-differentiation process of neural stem cells, and the shape was uniform (FIG. 1B). However, as shown in FIG. 1C, the mRNA expression levels of Nestin, Sox1, Pax6, Musashi-1, Vimentin, Olig2, Nkx2.1, FoxG1, Tuj1, and Ascl1, which are all molecular markers for neural stem cells showed an increase both under the condition treated with a small molecule inhibitor and under the condition not treated with a small molecule inhibitor. However, it was confirmed that the group treated with a small molecule inhibitor showed a significant increase in the expression of molecular markers for neural stem cells compared to the group not treated with a small molecule inhibitor (FIG. 1C). Additionally, the quantification of induced neural stem cells (iNSCs) was performed by flow cytometry of neural cell adhesion molecules (NCAMs) using a fluorescent flow cytometer (FACS Caliber) (FIG. 1D).

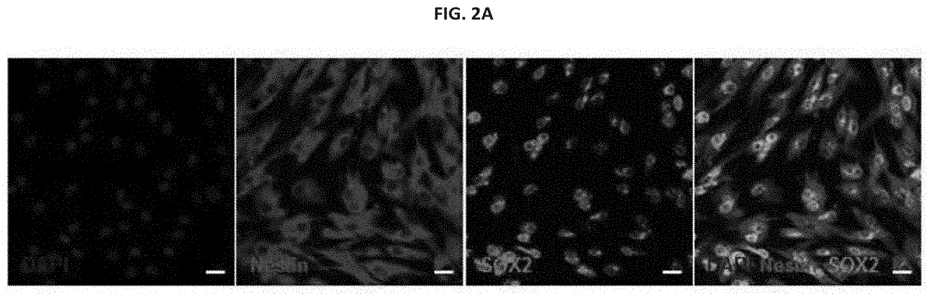

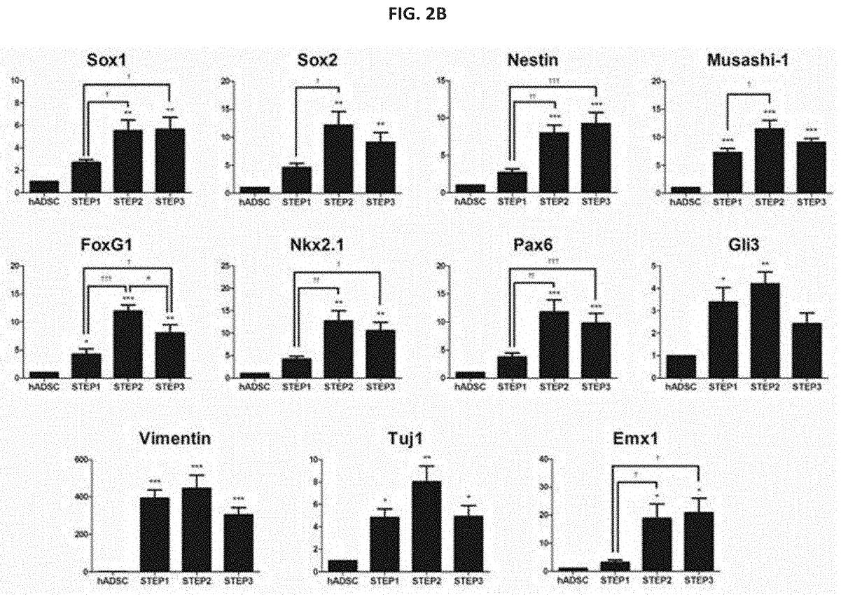

Additionally, as shown in FIGS. 2A to 2B, the detection of induced neural stem cells which express both Nestin and Sox2 were confirmed by immunofluorescence staining (FIG. 2A), and additionally, the changes in expression of the molecular markers (Sox1, Sox2, Nestin, Musashi-1, FoxG1, Nkx2.1, Pax6, Gli3, Vimentin, Tuj1, and Emx1) for the neural stem cells and early neurons were confirmed according to the cross-differentiation process of neural stem cells via real-time PCR analysis (FIG. 2B).

### <Experimental Example 2> Confirmation of Establishment of Differentiation Method from Adipose Tissue-Derived Stem Cells into Mature Neurons

The method of differentiating the adipocyte-derived mesenchymal stem cells in <Experimental Example 1> into neural stem cells was carried out in a three-step differentiation method, and as a result, cells having characteristics similar to those of neural stem cells were heterogeneously present, and for the differentiation of these cells into mature neurons, the following experiment was performed.

Specifically, adipocyte-derived mesenchymal stem cells (2.0×10⁵) were added into a gelatin-coated dish (6 cm²) and cultured in an incubator (37° C., 5% CO₂) for one day. On the next day, the medium was replaced with a medium, in which 10 μM SB431542, 0.1 μg/mL Noggin, and 0.5 μM LDN193289 (i.e., small molecule inhibitors) were added to a pre-induction medium, which contained 3% knockout serum replacement (KOSR), 1% penicillin/streptomycin, 1% glutamax, 3 mM D-glucose, 1% non-essential amino acids, and 4.0 ng/mL bFGF to the base medium (DMEM F-12), and the stem cells were cultured in an incubator (37° C., 5% CO₂) for 6 days. In a second step, the medium was replaced with a neural induction medium in which 2% B27 and 1% N2 were contained, and the stem cells were cultured in an incubator (37° C., 5% CO₂) for 5 days. Finally, in a third step, to increase the number of neural stem cell-like cells, the medium was replaced with a growth medium, in which bFGF (20 ng/mL) and epidermal growth factor (EGF; 20 ng/mL) were contained to the neural induction medium, and the stem cells were cultured in an incubator (37° C., 5% CO₂) for 5 days. Then, for differentiation into mature neurons, the cells which underwent these three-steps were washed with PBS, treated with 1× TryPLE select in an incubator at 37° C. for 3 to 4 minutes, and ground into single cells. In addition, the cells (1.0×10⁶) were added into a dish (35 mm²) coated in advance with PLO/FN and the cells (1.0×10⁵) were added into a 4-well dish, in which coverslips (12 mm²) were included, and stabilized in an incubator (37° C., 5% CO₂) for one day. On the next day, the medium was replaced with a medium, in which 1.0 μM purmorphamine and 10 ng/mL brain-derived neurotrophic factor (BDNF) were contained, and the cells were cultured in an incubator (37° C., 5% CO₂) for 12 to 14 days while replacing the medium with a fresh medium once every three days.

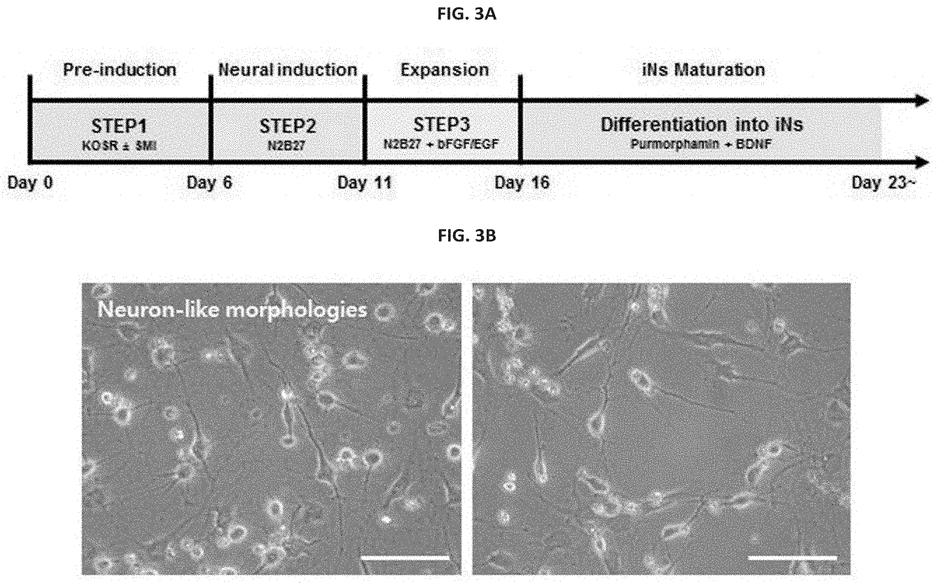

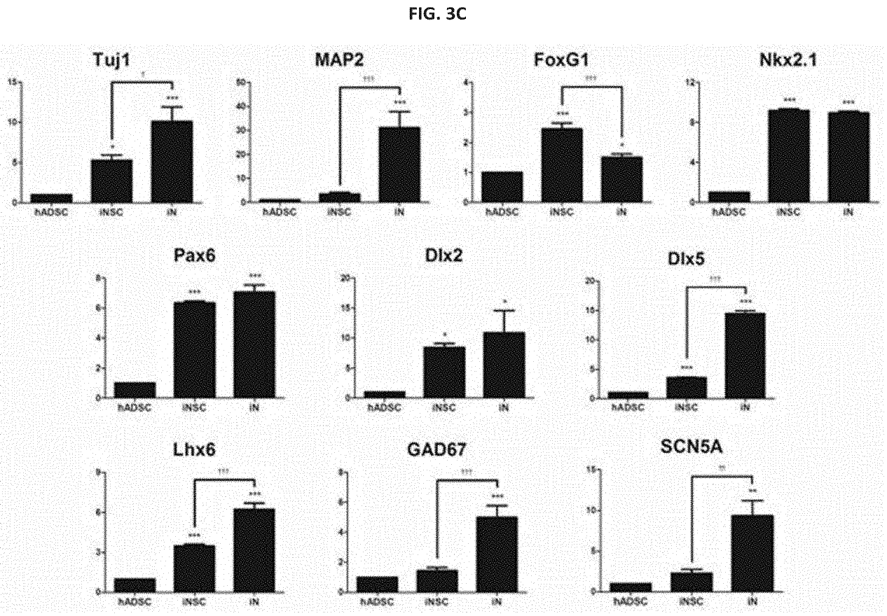

As a result, as shown in FIGS. 3A to 3C, induced neurons displayed a mature neuron-like morphology unlike the bipolar or multipolar neurites outgrowing from small cell bodies (FIG. 3B).

Additionally, quantitative analysis was performed via real-time PCR to examine the changes in expression levels of molecular markers for neurons (i.e., Tuj1 and MAP2), transcription factors associated with medial ganglionic eminence (MGE) expressed at early and late stages of development (i.e., FoxG1, Nkx2.1, Pax6, Dlx2, Dlx5, and Lhx6), a molecular marker for early GABA (i.e., GAD67), and a sodium ion channel gene (i.e., SCN5A), and it was confirmed that the expression of all of the genes associated with mature neurons in the induced neurons was significantly increased (FIG. 3C).

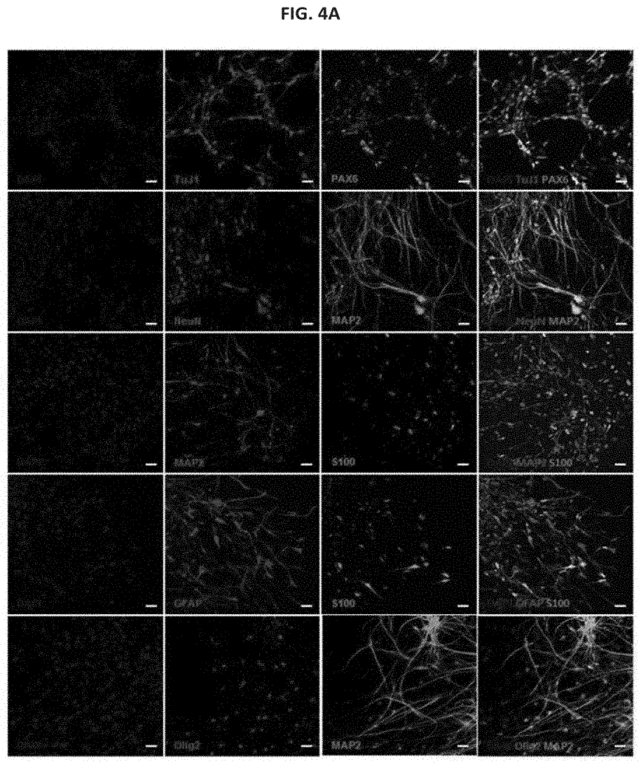

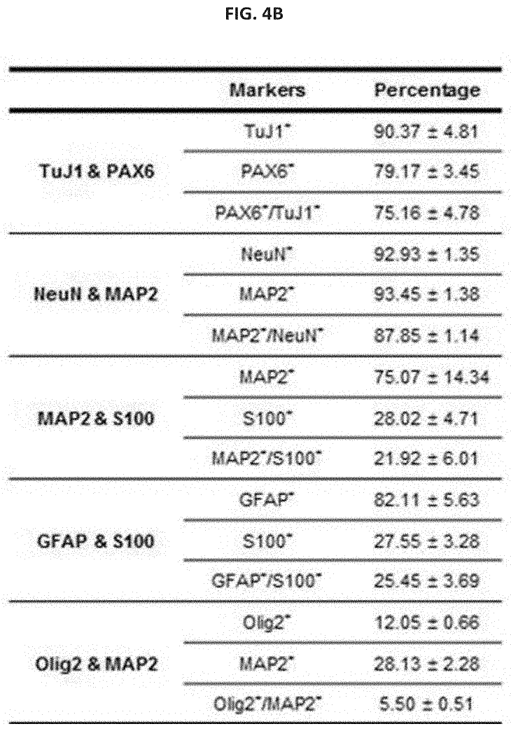

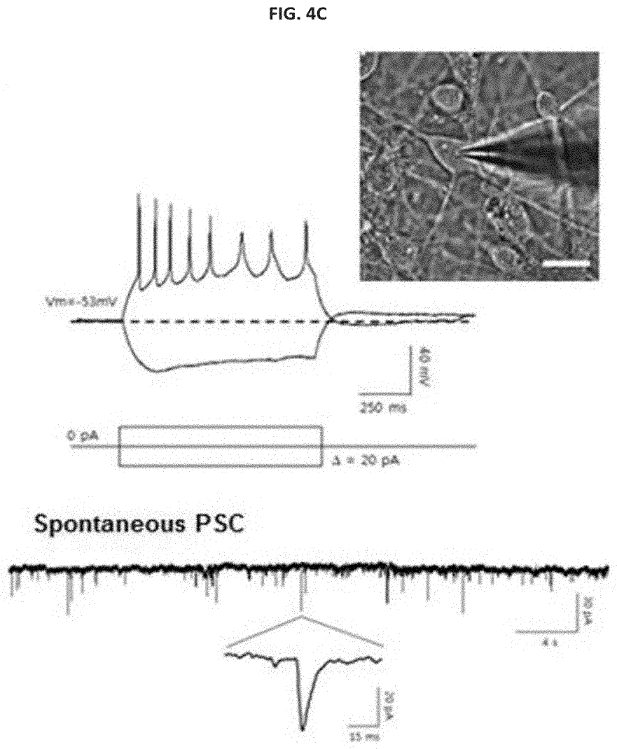

Additionally, as shown in FIGS. 4A to 4C, it was confirmed that neuronal molecular markers associated with brain development, such as molecular markers for neuronal progenitor cells (TuJ1, Pax6), molecular markers for mature neurons (NeuN/MAP2), molecular markers for astrocytes (GFAP/S100), and molecular markers for early oligodendrocytes (OLIG2) (FIG. 4A) have been expressed in the induced neurons, and the ratio of the number of the induced neurons that express these molecular markers alone or in common was quantitatively confirmed (FIG. 4B). Additionally, FIG. 4C illustrates samples of electrophysiological recordings measured from the induced neurons with typical neuronal morphology, sample images and the induction of action potential by current injection are indicated. The protocol for current injection is below the action potential trace. The protocol for current injection is below the action potential trace and the bottom trace is representative spontaneous synaptic activity obtained from induced neural cells in voltage clamp mode (holding at −60 mV). The magnified single current is shown below the continuous trace (FIG. 4C).

### <Experimental Example 3> Confirmation of Establishment of Differentiation Method from Adipose-Derived Stem Cells into GABAergic Neurons

Likewise, a method for differentiating adipocyte-derived mesenchymal stem cells into GABAergic neurons was established based on the three-step differentiation method established in <Experimental Example 1>. To increase the number of days for culturing in a neuronal mature medium, the number of days for cross-differentiation of neural stem cells was shortened, and subsequently, a medium for inducing neurons consisting of purmorphamine and BDNF and a medium for maturing neurons consisting of dbcAM and BDNF were used.

Specifically, adipocyte-derived mesenchymal stem cells (2.0×10⁵) were added into a gelatin-coated dish (6 cm²) and cultured in an incubator (37° C., 5% CO₂) for one day. On the next day, the medium was replaced with a medium, in which 10 μM SB431542, 0.1 μg/mL Noggin, and 0.5 μM LDN193289 (i.e., small molecule inhibitors) were added to a pre-induction medium, which contained 3% KOSR, 1% penicillin/streptomycin, 1% glutamax, 3 mM D-glucose, 1% non-essential amino acids, and 4.0 ng/mL bFGF to the base medium (DMEM F-12), and the stem cells were cultured in an incubator (37° C., 5% CO₂) for 6 days. After completion of the first step, the cells were washed with PBS, treated with 1× TryPLE select in a 37° C. incubator for 3 to 4 minutes, and well ground into single cells. Then, the cells were divided such that the cells were added into a PLO/FN-coated dish (35 mm²) at a density of 1.0×10⁶ cells/well while adding to a PLO/FN-coated 4-well dish, in which a coverslips (12 mm²) are included, at a density of 1.0×10⁵ cells/well, and stabilized by incubating in an incubator (37° C., 5% CO₂) for one day. In a second step, the medium was replaced with a neural induction medium in which 2% B27 and 1% N2 were contained, and the cells were cultured in an incubator (37° C., 5% CO₂) for 5 days. Then, for differentiation into GABAergic neurons, the medium was replaced with a medium, in which 1.0 μM purmorphamine and 10 ng/mL BDNF were contained, and the cells were cultured in an incubator (37° C., 5% CO₂) for 10 to 14 days while replacing the medium with a fresh medium once every three days in the same manner. After the differentiation into neurons for 10 to 14 days, to induce a further differentiation into GABAergic neurons, the medium was replaced with a medium in which 1 μM purmorphamine and 10 ng/mL BDNF were contained, and 50 μM dbcAMP and 20 ng/mL BDNF were added to the medium for inducing neurons, and the cells were further differentiated for 13 to 20 days.

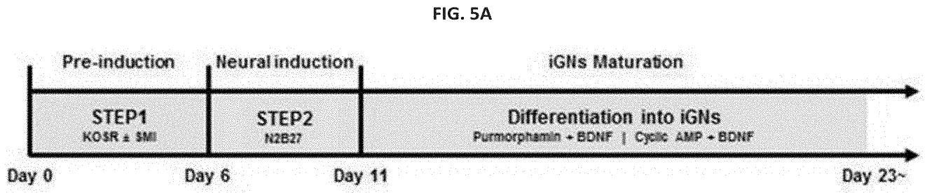

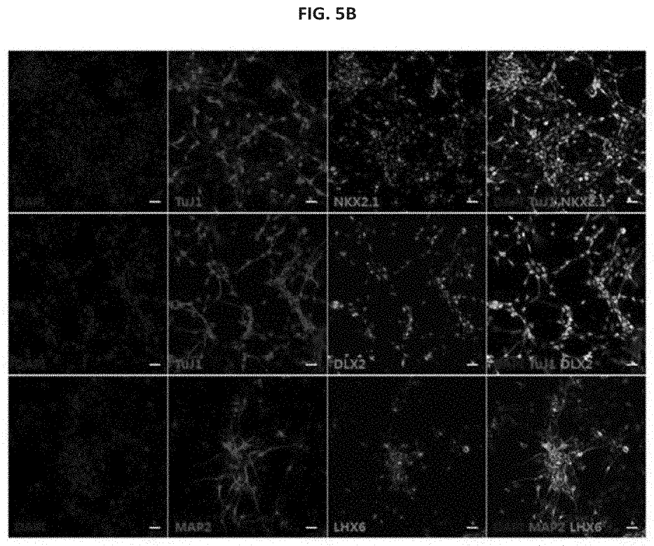

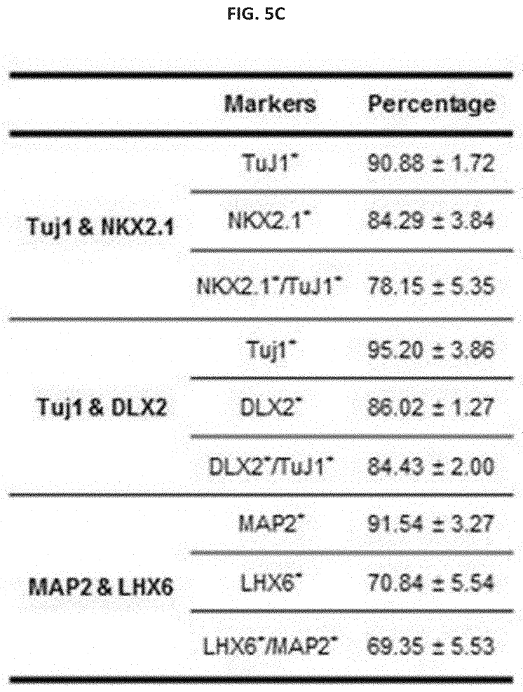

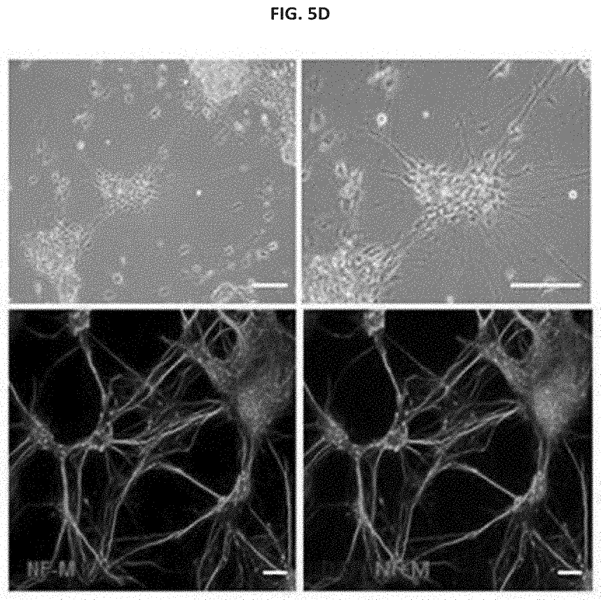

As a result, as shown in FIGS. 5A to 5D, it was confirmed that induced GABAergic neurons displayed a number of radial neurites extending from a neurosphere-like cell cluster, and many of the induced GABAergic neurons expressed NKX2.1, DLX2, and LHX6, which are molecular markers for medial ganglionic eminence (MGE), and TuJ1 and MAP2, which are molecular markers for neurons (FIG. 5B), and the ratio of the number of induced GABAergic neurons which express these molecular markers alone or in common were quantitatively confirmed (FIG. 5C). Additionally, it was confirmed that the induced GABAergic neurons displayed neurites extending from a neurosphere-like cell cluster and that the induced GABAergic neurons have expressed neurofilament-M (NF-M) (FIG. 5D).

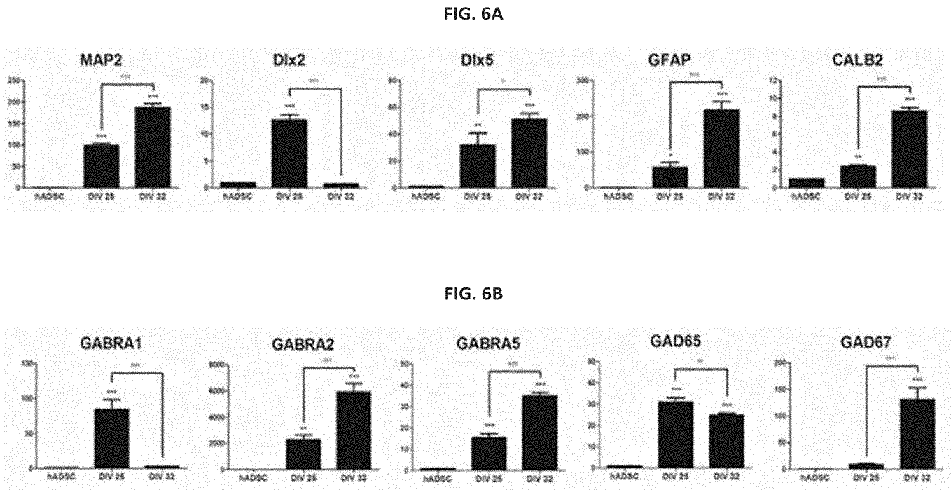

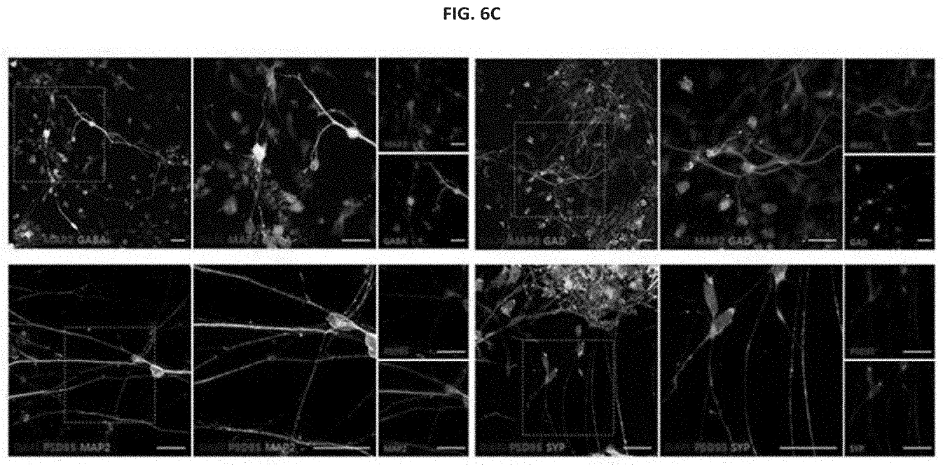

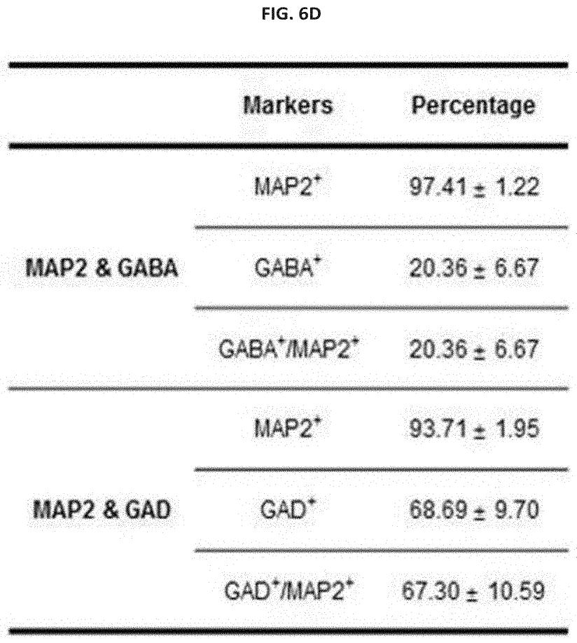

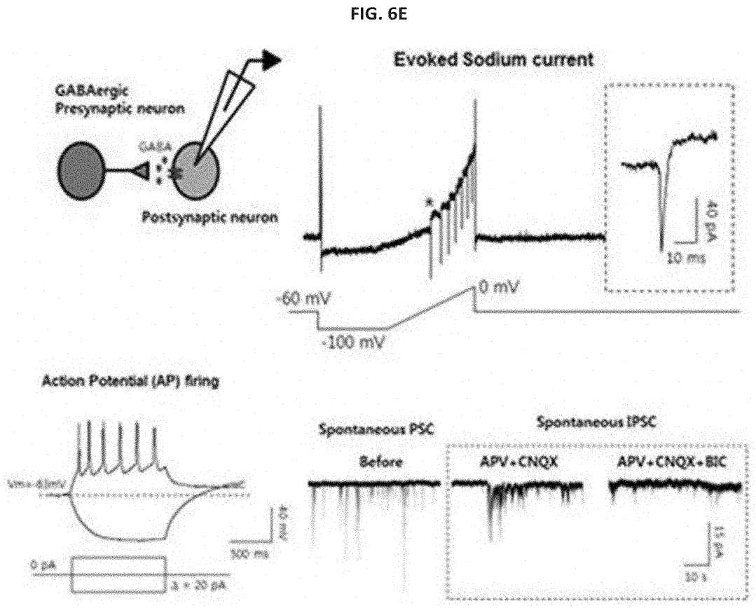

Additionally, as shown in FIGS. 6A to 6E, the real-time PCR analysis revealed the changes in gene expression of neurons (MAP2), medial ganglionic eminence (MGE) transcription factors (Dlx2, Dlx5), astrocytes (GFAP), a calcium binding protein (CALB2), GABA receptors (GABRA1. GABRA2, and GABRA5), and GABA (GAD65, GAD67). While most of the induced GABAergic neurons with longer differentiation days being on the 32^{nd} day after differentiation in a test tube culture showed a dramatic increase of gene expression of more mature GABAergic neurons compared to the induced GABAergic neurons being on the 25^{th} day after differentiation in a test tube, these induced GABAergic neurons with longer differentiation days being on the 32^{nd} day after differentiation showed a relative decrease in the gene expression of the GABAergic neurons at the early stage (FIGS. 6A and 6B). Additionally, it was confirmed via immunofluorescence staining that all of the molecular markers for GABAergic neurons and mature neurons (GABA/MAP2, GAD/MAP2, PSD95/MAP2, and PSD95/SYP) were expressed (FIG. 6C), and this was quantitatively confirmed (FIG. 6D). Additionally, as shown in the bottom right of FIG. 6E, it was confirmed that a spontaneous inhibitory post-synaptic current (IPSC) appears while being with a glutamate receptor blocker (50 μM APV and 20 μM CNQX), and the IPSC disappears when treated with 10 μM bicuculline, which is a GABA_{A} receptor blocker (FIG. 6E).

### <Experimental Example 4> Induction of Differentiation from Human Bone Marrow-Derived Mesenchymal Stem Cells into Growth Factor-Releasing Human Mesenchymal Stem Cells (gfMSCs)

The present inventors induced the differentiation of the human bone marrow-derived mesenchymal stem cells (hMSCs), which were purchased from Cambrex (USA) into human growth factor-releasing human mesenchymal stem cells (gfMSCs).

Specifically, undifferentiated human bone marrow-derived mesenchymal stem cells (untreated hMSCs; uhMSCs) were cultured in a cell incubator (37° C., 5% CO₂) along with a base growth medium consisting of a DMEM-low glucose medium, 10% FBS, and 1% penicillin/streptomycin mixture. After culturing the cells in the cell incubator for two days, the base growth medium was replaced with a primary differentiation medium [(a DMEM-low glucose medium, 10% FBS, 1% penicillin/streptomycin mixture, 1.0 mM (β-mercaptoethanol)] and the cells were cultured for 24 hours. Then, the mesenchymal stem cells were washed with PBS and cultured after replacing the medium with a secondary differentiation medium (a DMEM-low glucose medium, 10% FBS, 1% penicillin/streptomycin mixture, 0.28 μg/mL all-trans-retinoic acid). After three days, the mesenchymal stem cells were washed with PBS and the medium was replaced with a tertiary differentiation medium (a DMEM-low glucose medium, 10% FBS, 1% penicillin/streptomycin mixture, 10 μM forskolin, 10 ng/mL human basic-fibroblast growth factor, 5.0 ng/mL human platelet-derived growth factor-AA, and 200 ng/mL heregulin-β1) and cultured for 8 days. In particular, the tertiary differentiation medium was replaced once every two days. The morphology of the hMSCs under cultivation for differentiation were observed under a microscope. The survival rate of the cells during the process of inducing differentiation was confirmed via trypan blue staining and measured as a percentage of the cells which were not stained with trypan blue (living cells).

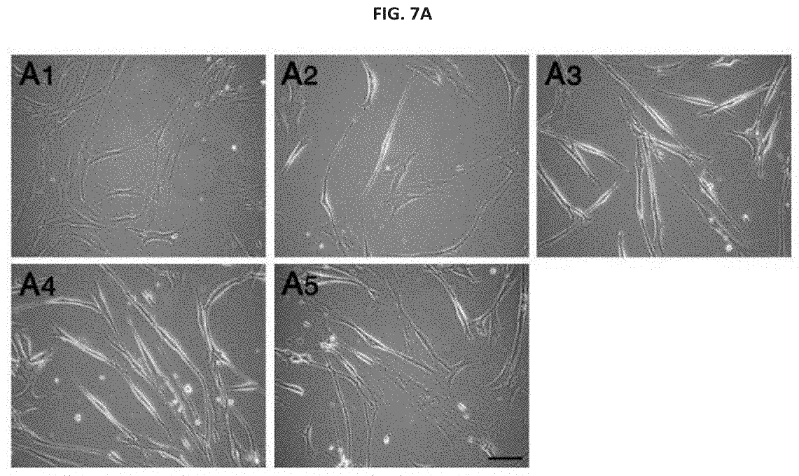

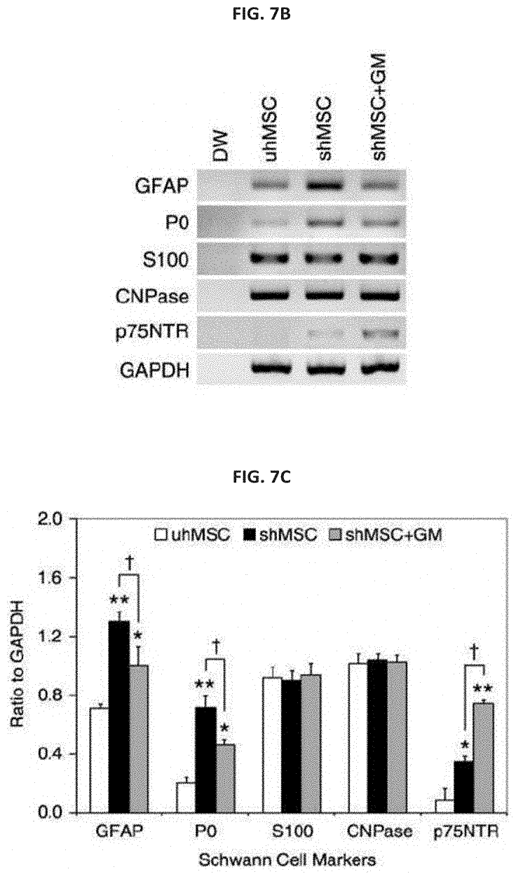

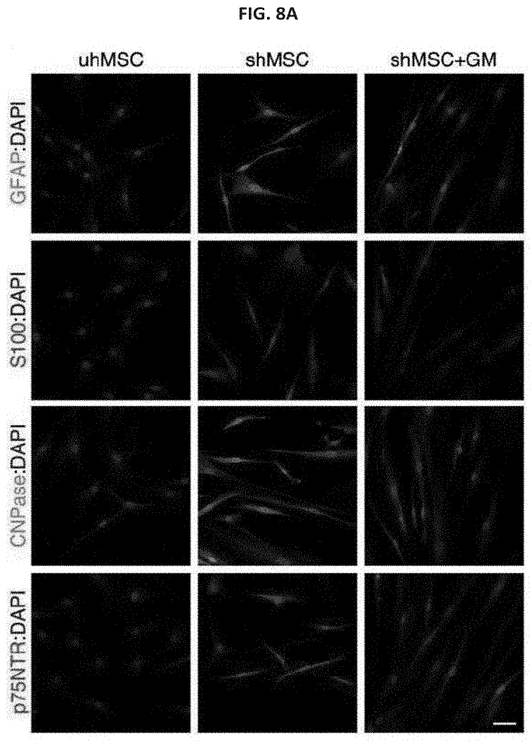

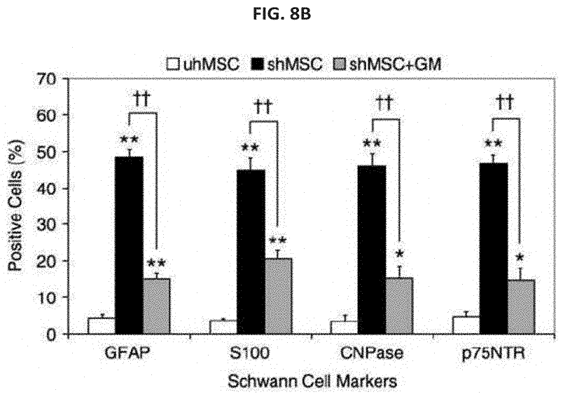

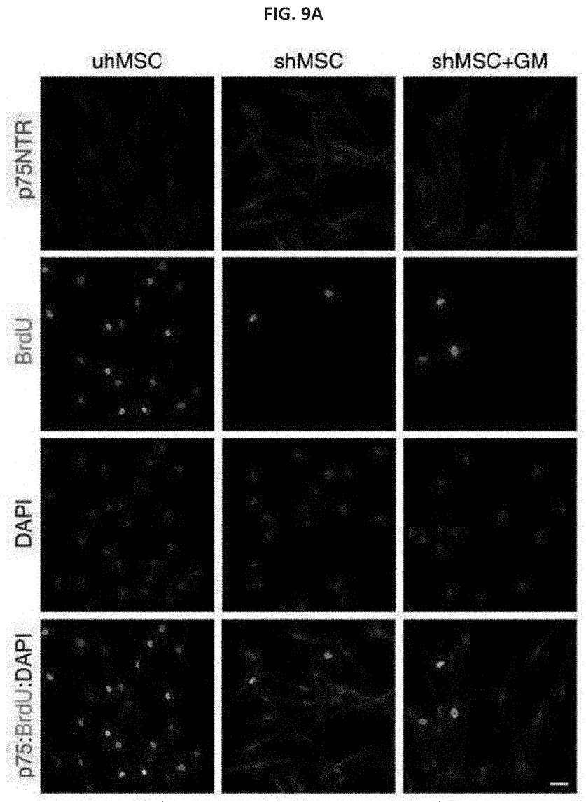

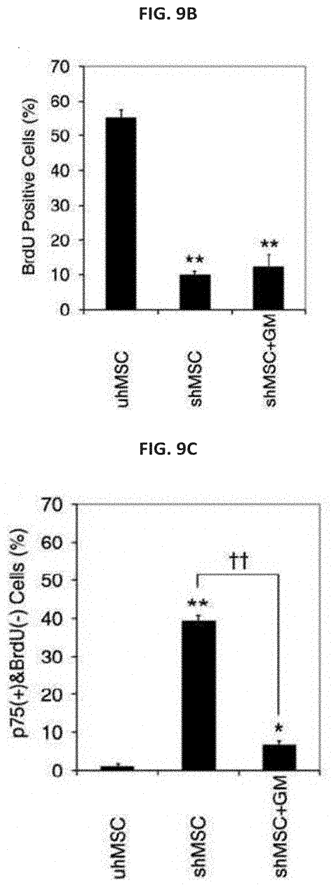

As a result, as shown in FIG. 7A, it was observed through the process of induction of cell differentiation that the uhMSCs were changed into an elongated bipolar shape of human stem cells that hypersecrete growth factors over time. In particular, it was observed that the uhMSCs were changed such that 54% of the uhMSCs were changed into growth factor-releasing human mesenchymal stem cells (gfMSCs) on the 8^{th} day of differentiation and 80% or more of the uhMSCs were changed into gfMSC on the 12^{th} day of differentiation, and that most cells were alive during the process of inducing the differentiation. It was confirmed that about 50% of the stem cells cultured in a common growth medium maintained the shape of growth factor-releasing human mesenchymal stem cells (gfMSCs) for three days after completion of the induction of differentiation.

### <Experimental Example 5> Confirmation of Secretion of Growth Factors by Differentiated Human Stem Cells that Hypersecrete Growth Factors

<5-1> Analysis of Secretion of Growth Factors by Differentiated Human Stem Cells that Hypersecrete Growth Factors

The present inventors analyzed the human growth factors secreted in the gfMSC cells differentiated in Example 4.

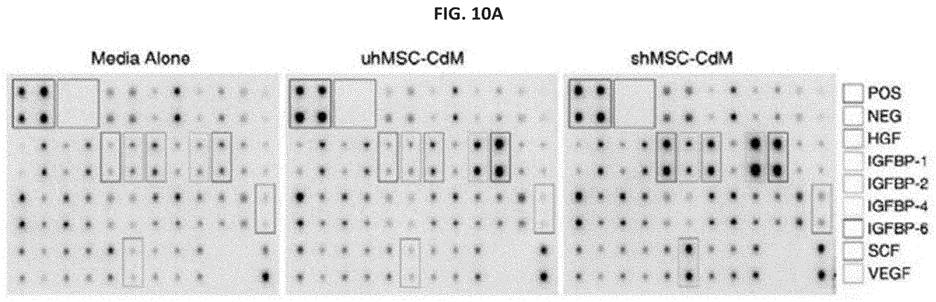

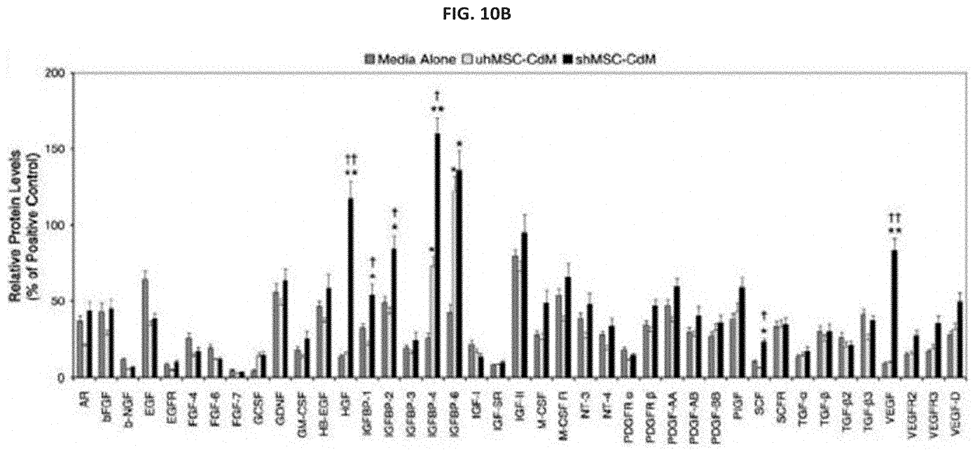

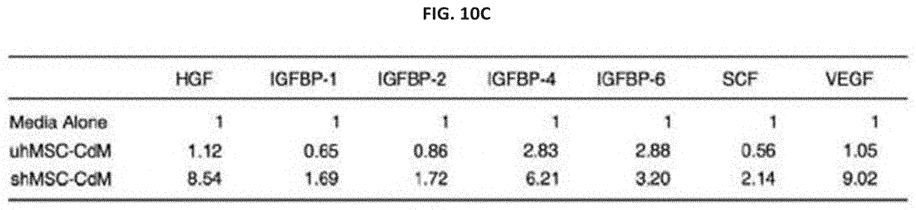

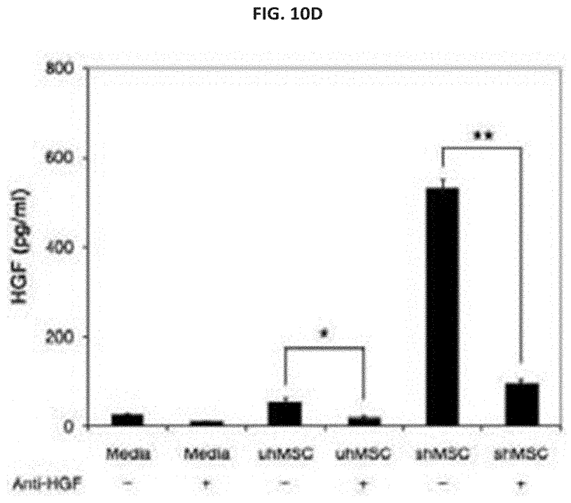

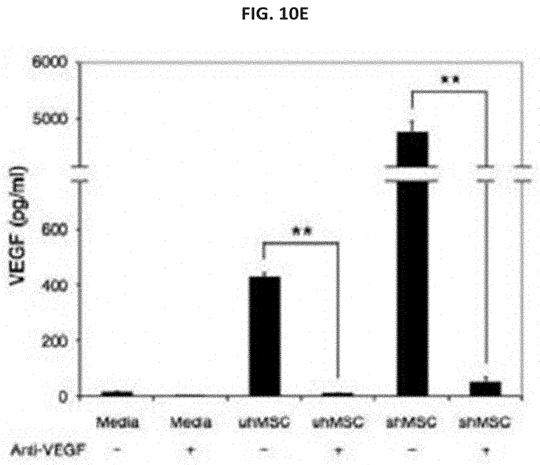

Specifically, to prepare a conditional medium (CdM), uhMSCs and gfMSCs were washed 4 times with PBS and cultured in a serum-free Neurobasal medium (Gibco-BRL). After 18 hours of incubation, each medium was collected, centrifuged at 1,500 g for 5 minutes to remove residuals of cells. After centrifugation, the supernatant (uhMSC-CdM and gfMSC-CdM) of each cell culture fluid obtained therefrom was analyzed using the human growth factor array (RayBiotech) according to the manufacturer's guidelines. In particular, the medium used in the above cell culture as a negative control group was also measured. In brief explanation, the human growth factor array membrane was blocked with a blocking buffer and the conditional medium (1.0 mL) was cultured at 4° C. overnight, and the membrane was washed at room temperature three times with washing buffer I and twice with washing buffer II for 5 minutes, respectively. Then, diluted biotin-binding antibodies (1.0 mL) were added thereto, cultured at room temperature for two hours, and the membrane was washed again, and finally, cultured at room temperature along with diluted HRP-binding streptavidin for two hours. The membrane was detected using LAS-3000 (Fujifilm), which is Lumino image analyzer, and the signal intensities were quantified using a densitometer (Bio-Rad). The relative expression level of each human growth factor was analyzed by comparing the staining intensities of each slot relative to the staining intensity of positive control group (POS) which is in the results of the same analysis. The analysis results are illustrated in FIG. 10B (the secretion rates (%) of growth factors of uhMSC-CdM or gfMSC-CdM relative to the positive control group) and FIG. 10C (the amount of secretion (fold) of growth factor of uhMSC-CdM or gfMSC-CdM relative to the negative control group). The above experiment was repeated independently at least three times and the results were indicated as mean±SEM.

As a result, as shown in FIGS. 10A to 10C, it was observed that hepatocyte growth factor (HGF), vascular endothelial growth factor (VEGF), insulin-like growth factor binding protein1 (IGFBP-1), IGFBP-2, IGFBP-4, IGFBP-6, and stem cell factor (SCF) showed greater expression levels in gfMSC-CdM than in uhMSC-CdM, and these results are shown in rectangles.

<5-2> Analysis of Production Levels of HGF and VEGF by Differentiated Human Stem Cells that Hypersecrete Growth Factors

The present inventors confirmed the differences in the amount of secretion of growth factors in uhMSCs and gfMSCs confirmed in Example 5-1 via enzyme-linked immunosorbent assay (ELISA).

ELISA was performed using the ELISA Kit (RayBiotech) for HGF or VEGF in groups where the medium, uhMSC-CdM or gfMSC-CdM were treated or untreated with anti-HGF antibodies [mouse anti-human HGF (MAB294), R&D Systems] or anti-VEGF antibodies [mouse anti-human VEGF antibody (MAB293), R&D Systems]. The above experiment was repeated independently at least three times and the results were indicated as mean±SEM.

As a result, as shown in FIGS. 10D and 10E, it was confirmed that the amounts of HGF or VEGF produced in gfMSCs were increased by about 10-fold and about 11-fold compared to that produced in uhMSCs.

<5-3> Analysis of Production Levels of HGF and VEGF by Differentiated Human Stem Cells that Hypersecrete Growth Factors Per Culture

In order to determine how long productions of HGF and VEGF are maintained after completion of induction of differentiation, the present inventors cultured gfMSCs in an induced differentiation medium (SCIM) and a common growth medium (GM) for 0, 1, 3, and 5 days, respectively, and performed ELISA assay using in the same manner as in Example 5-2.

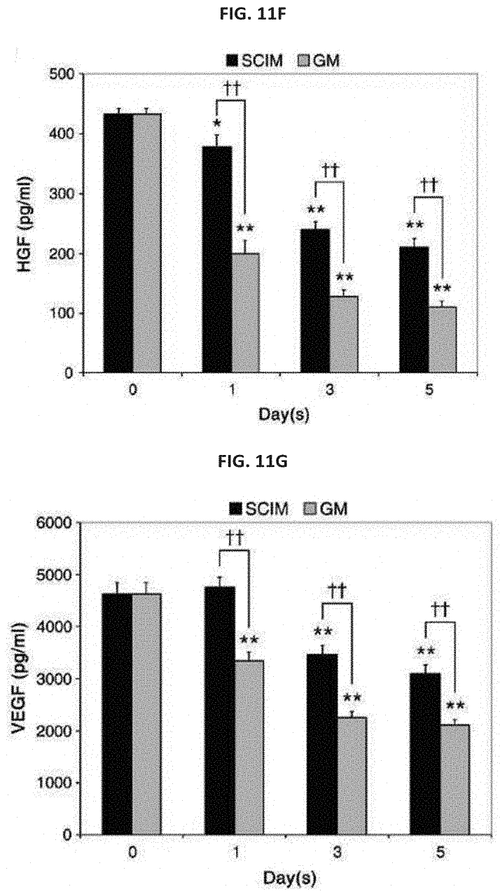

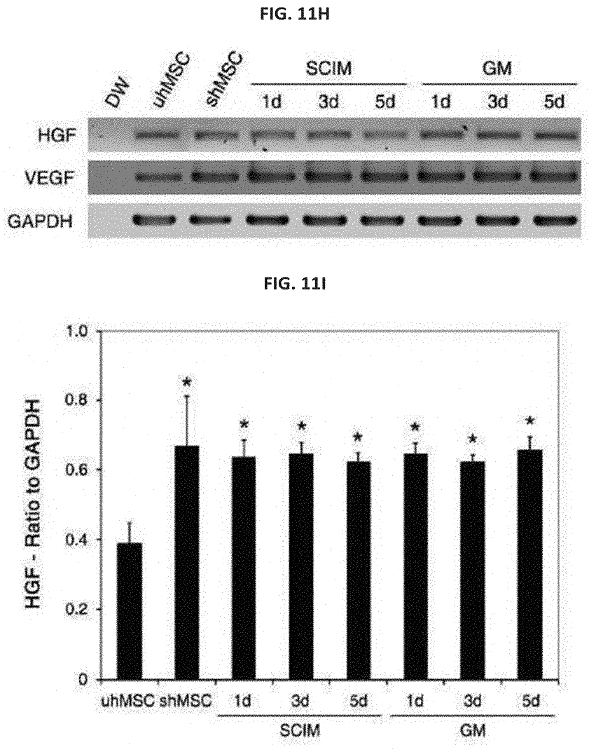

As a result, as shown in FIGS. 11F and 11G, it was confirmed that although the amounts of secretion of HGF and VEGF were decreased over time, the amounts of secretion were still maintained at a level higher than that of uhMSCs (Control), and that the amounts of secretion were higher in an induced differentiation medium (SCIM) than in a common growth medium (GM).

<5-4> Analysis of mRNA Expression Levels of HGF and VEGF by Differentiated Human Stem Cells that Hypersecrete Growth Factors

The present inventors have confirmed whether the expression of growth factors according to the differentiation of uhMSCs confirmed in Examples 5-2 and 5-3 are controlled from the mRNA stage via quantitative RT-PCR (qRT-PCR). Specifically, water, uhMSC, gfMSCs, or gfMSCs were subjected to qRT-PCR in groups cultured each in SCIM or GM for 1, 3, and 5 days, using a primer pair for HGF consisting of a sense primer (5′-atgctcatggaccctggt-3′) of SEQ ID NO: 53 and an antisense primer (5′-gcctggcaagcttcatta-3′) of SEQ ID NO: 54 and a primer pair for VEGF consisting of a sense primer of SEQ ID NO: 55 (5′-gccttgctgctctacctcca-3′) and an antisense primer of SEQ ID NO: 56 (5′-caaggcccacagggatttt-3′) in the same manner as in Example 4-2 above.

As a result, as shown in FIGS. 11H to 11J, it was confirmed that the mRNA expression levels of HGF and VEGF in gfMSCs were maintained at high levels compared to that of uhMSCs until the 5^{th} day, regardless of the type of medium.

### <Experimental Example 6> Effects of Differentiated Human Stem Cells that Hypersecrete Growth Factors on Neurons

The present inventors performed the following experiment to examine the effects of the gfMSCs differentiated from uhMSCs that hypersecrete growth factors on neurons.

<6-1> Confirmation of Increase in Neurite Growth by Differentiated Human Stem Cells that Hypersecrete Growth Factors

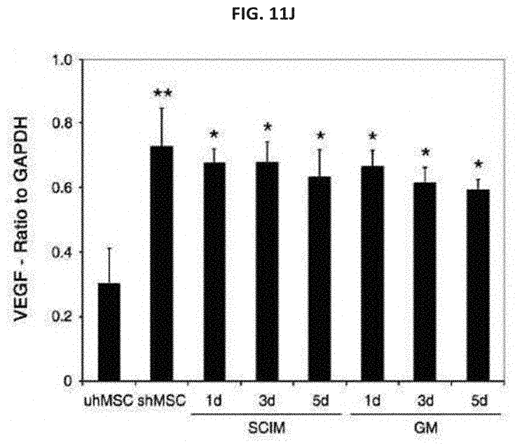

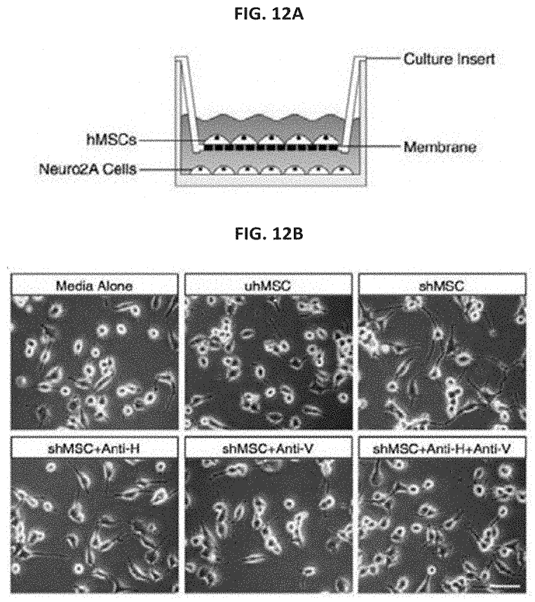

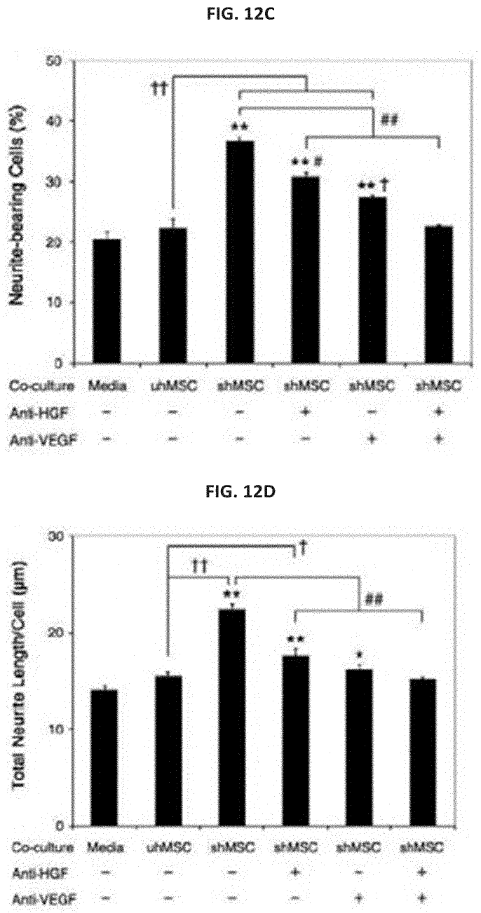

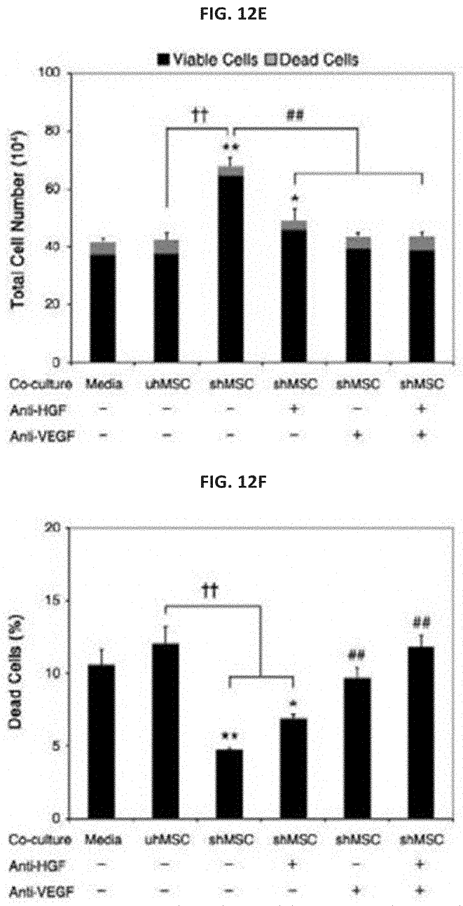

Neuro2A cells (ATCC #CCL-131), which are neuroblastoma, were plated into a 6-well plate coated with fibronectin (2 μg/mL) at a density of 1.3×10⁵ cells/well and cultured in a growth medium overnight. In addition, the Neuro2A cells were cultured in a Neurobasal medium (0.5% FBS), which was either treated or untreated with anti-HGF antibodies or anti-VEGF antibodies. 24 Hours before plating the Neuro2A cells, uhMSCs and gfMSCs were plated onto a cell culture insert with a size of 1.0-μm diameter at a density of 5×10⁴ cells/insert and cultured in each medium. After 48 hours, the culture insert was washed three times with PBS and then transferred into a 6-well plate where Neuro2A cells were included (FIG. 12A). In the case of a negative control well where no cells are included, only the culture insert where only 0.5% FBS-containing Neurobasal medium was contained was included, and cultured in the same condition as in the experimental group. After culturing for 48 hours the uhMSCs or gfMSCs transferred into a 6-well plate where Neuro2A cells were included, the Neuro2A cells were analyzed using a phase contrast microscope. In addition, as a result of the above analysis, it was confirmed that the ratio of the cells having neurites with a length greater than the diameter of at least one cell body and the sum of the neurites per cell in Neuro2A cells are shown in FIG. 12C and FIG. 12D, respectively.

As a result, as shown in FIGS. 12B to 12D, when compared with the uhMSCs, the length of neurites was longer in Neuro2A cells which were co-cultured with gfMSCs, and it was confirmed that such an effect was decreased by antibodies for HGF and/or VEGF.

<6-2> Confirmation of Increase in Neuron Growth and Survival by Differentiated Human Stem Cells that Hypersecrete Growth Factors

The effects of gfMSCs on the growth and survival of neurons in the same conditions as in Example 6-1 were confirmed via the trypan blue staining method. For each group, the average number of the Neuro2A cells and the ratio of the apoptosized Neuro2A cells are shown in FIG. 12E and FIG. 12F, respectively. The cell death was indicated as a percentage of the cells stained with trypan blue (apoptosized cells). Per each group, at least 700 cells were selected from any region and analyzed, and the experiment was repeated independently at least three times and the results are indicated as mean±SEM.

As a result, as shown in FIGS. 12E and 12F, when compared with the uhMSCs, the effects of increasing the growth of Neuro2A cells which were co-cultured with gfMSCs and decreasing cell death were confirmed, and it was confirmed that such effects were decreased by the antibodies for HGF and/or VEGF.

### <Experimental Example 7> Effects of Differentiated Human Stem Cells that Hypersecrete Growth Factors on Damaged Spinal Cord Tissue Sections

<7-1> Confirmation of Increasing Growth of Neurites in Damaged Spinal Cord Tissue by Differentiated Human Stem Cells that Hypersecrete Growth Factors

In order to confirm whether the effect of gfMSC of increasing the growth of neurites in neurons confirmed in Example 3-1 and Example 6-2 is exhibited in the same manner ex vivo, the present inventors performed the following experiment with the approval of Experimental Animal Steering Committee of Seoul National University.

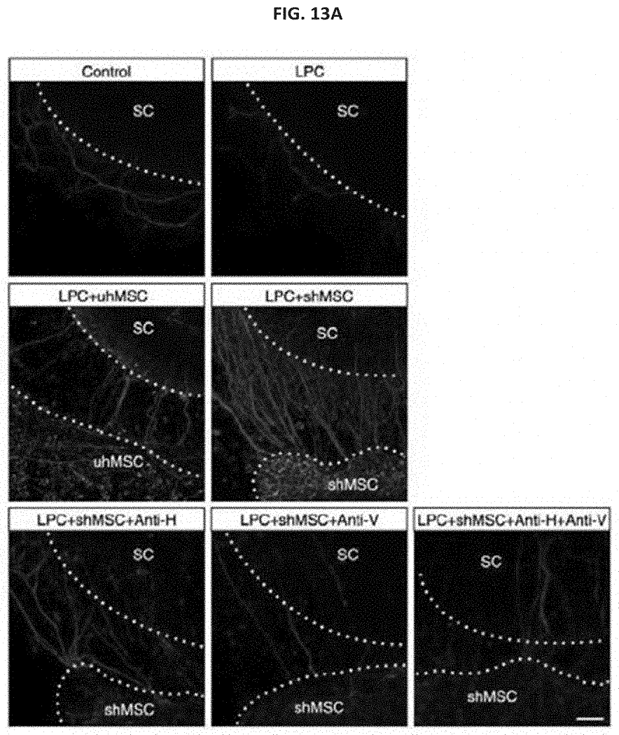

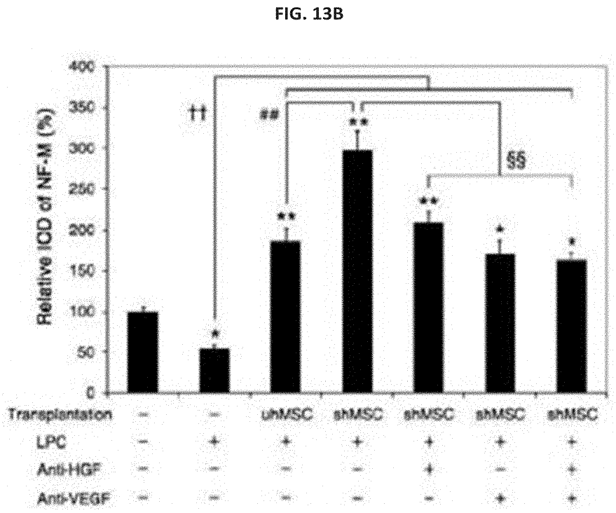

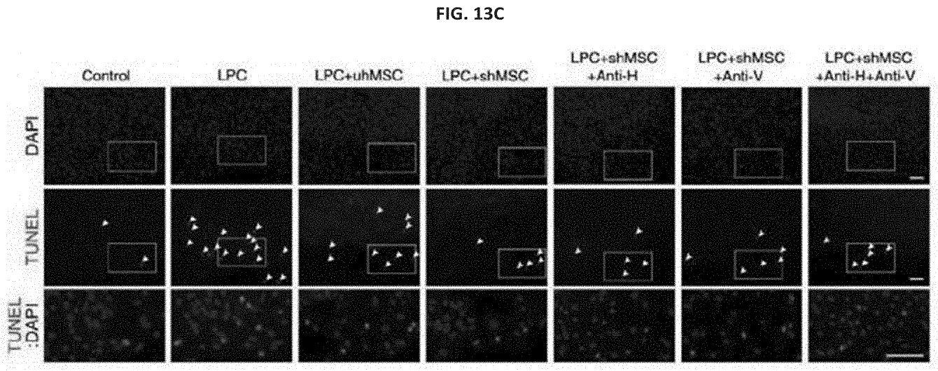

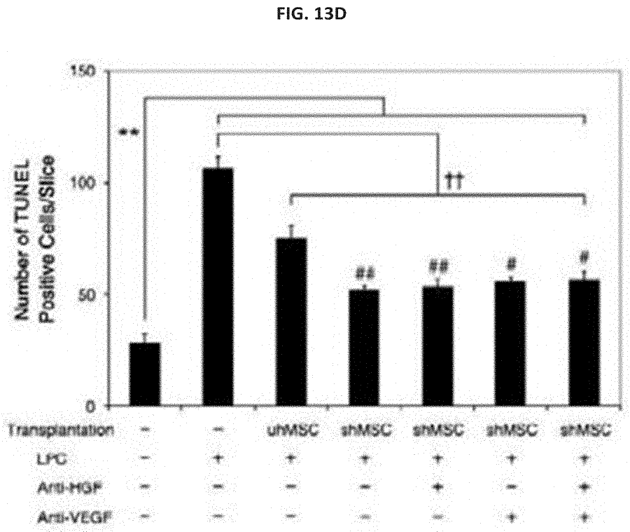

Specifically, spinal cord tissue sections of 16-day-old Sprague-Dawley were anesthetized with avertin and lumbar spinal cord of the rats were collected under sterile conditions. In the spinal cord, nerve roots and connective tissues were removed using the Hank's balanced salt solution (Gibco-BRL) containing cold glucose (6.4 mg/mL), and the spinal cord was cut into sections with a thickness of 400 μm using the McIlwain tissue chopper (Mickle Laboratory Engineering) and then demyelinated lysolecithin-treated slices (LPC) were prepared. Four sections were carefully placed on a single membrane insert (Millicell-CM; Millipore), and then, the membrane insert was placed on a 6-well plate where 1 mL of a medium containing 50% Eagle's minimum essential medium (Gibco-BRL), 25% Hank's balanced salt solution, 25% horse serum (Gibco-BRL), 6.4 mg/mL glucose, and 20 mM HEPES (Sigma-Aldrich) was included. The tissue sections were cultured in an incubator (37° C., 5% CO₂) and the culture medium was replaced twice per week. On the 7^{th} day of culture, the spinal cord tissue sections were treated with lysophosphatidyl choline (lysolecithin; 0.5 mg/mL) for 17 hours. Then, the medium was replaced with a fresh medium which was treated or untreated with anti-HGF antibodies or anti-VEGF antibodies. For the transplantation of mesenchymal stem cells, the uhMSCs or gfMSCs were treated with trypsin and centrifuged at 2,000 g for 3 minutes. The precipitated cells were suspended in PBS, and the cells (3×10⁴ cells/1.5 μL) were each transplanted directly into the ventral part of each spinal cord tissue section using an aspirator tube assembly to which a microcapillary pipet is connected. For the histoimmunofluorescence staining analysis, spinal cord tissue sections were fixed with 4% paraformaldehyde at 4° C. overnight and allowed to be permeabilized by treating with PBS containing 0.5% Triton X-100 and 1% bovine serum albumin (BSA) at room temperature for 10 minutes. After blocking the tissue sections by culturing in PBS containing 0.1% Triton X-100 and 3% bovine serum albumin (BSA) for one hour, the tissue sections were cultured and stained at 4° C. using each of rabbit anti-neurofilament (NF)-M (1:500, staining of nerve fibers (Axons) of spinal cord tissue sections, Chemicon) and mouse anti-human nuclear antibodies (1:500, staining of transplanted human mesenchymal stem cells, Chemicon) overnight. The cultivation was performed in the same manner as described above by using Alexa 546 antibodies (red) as secondary antibodies for anti-NF-M antibodies and by using Alexa 488 antibodies (green) as secondary antibodies for anti-human nuclear antibodies. The z-stack images were captured using a laser-scanning confocal microscope at 3-4 μm intervals. The neurofilament-M immunofluorescence was observed through a z-stack laser-scanning confocal microscope, and quantified into a morphometric image analysis (integrated optical density, IOD) using the Image J software (National Institutes of Health). The results observed by the microscope are shown in FIG. 13A and the relative integrated optical density (IOD) of the nerve fibers stained with NF-M in the spinal cord tissue sections are shown in FIG. 13B. In FIG. 13A, the spinal cord tissue sections (SC) and transplanted mesenchymal stem cells are indicated by a boundary line. The integrated optical density was normalized by the spinal cord tissue of the control group, and the histograms represent mean±SEM for at least 7 tissue sections.

As a result, as shown in FIGS. 13A and 13B, when uhMSCs were transplanted into LPC, the growth of neurites showed a 3.5-fold increase and when gfMSCs were transplanted into LPC, the growth of neurites showed a 5.5-fold increase, compared to when LPC was present alone. In addition, it was confirmed that the growth of neurites in LPC by the transplantation of gfMSCs showed a significant decrease when treated with antibodies for HGF and/or VEGF.

<7-2> Confirmation of Inhibition of Cell Death in Damaged Spinal Cord Tissue by Differentiated Human Stem Cells that Hypersecrete Growth Factors

In order to confirm whether the effect of gfMSCs inhibiting cell death confirmed in Example 6-1 and Example 6-2 can act in the same way ex vivo, the present inventors performed the following experiment.

Specifically, demyelinated spinal cord tissue sections (LPC) were prepared in the same manner as in Example 7-1. Four sections were carefully placed on a single membrane insert (Millicell-CM; Millipore), and then, the membrane insert was placed on a 6-well plate where 1 mL of a medium containing 50% Eagle's minimum essential medium (Gibco-BRL), 25% Hank's balanced salt solution, 25% horse serum (Gibco-BRL), 6.4 mg/mL glucose, and 20 mM HEPES (Sigma-Aldrich) was included. The tissue sections were cultured in an incubator (37° C., 5% CO₂) and the culture medium was replaced twice per week. On the 7^{th} day of culture, the spinal cord tissue sections were treated with lysophosphatidyl choline (lysolecithin; 0.5 mg/mL) for 17 hours. Then, the medium was replaced with a fresh medium which was treated or untreated with anti-HGF antibodies or anti-VEGF antibodies. For the transplantation of mesenchymal stem cells, the uhMSCs or gfMSCs were treated with trypsin and centrifuged at 2,000 g for 3 minutes. The precipitated cells were suspended in PBS, and the cells (3.0×10⁴ cells/1.5 μL) were each transplanted directly into the ventral part of each spinal cord tissue section using an aspirator tube assembly to which a microcapillary pipet is connected. Terminal deoxynucleotidyltransferase dUTP nick-end labeling (TUNEL) staining (red) with regard to cell death mediated by lysolecithin in each group was performed according to the manufacturer's instructions using In Situ Cell Death Detection Kit (Roche Diagnostics). In particular, nuclei were observed by DAPI staining (blue). The above results were observed through a microscopic image and are shown in FIG. 13C (arrow head: TUNEL-positive cells), and the number of TUNEL-positive cells in the ventral part of the spinal cord tissue sections was measured from the 100× magnification microscopic image and the degree of cell death is shown as a graph in FIG. 13D. The histograms represent mean±SEM for at least 5 tissue sections.

As a result, as shown in FIGS. 13C and 13D, it was confirmed that the cell death of the spinal cord tissue sections with an increase in cell death by lysolecithin treatment was further decreased in the case where gfMSCs were transplanted into LPC, when compared to the case where uhMSCs were transplanted into LPC. However, no significant difference was shown with regard to the effect of inhibiting cell death of spinal cord tissue sections of the gfMSCs according to antibody treatment for HGF and/or VEGF.

### <Experimental Example 8> Effects of HGF and VEGF on Neurons and Damaged Spinal Cord Tissue

The present inventors, to confirm whether the effects of increasing cell growth and neurites in the neurons of gfMSCs, growth of neurites in damaged spinal cord tissue sections, and inhibition of cell death as confirmed in Examples 6-1 to 7-2 are due to the growth factors secreted by gfMSC, the present inventors performed the following experiment.

<8-1> Confirmation of Increase of Neurite Growth in Neurons by HGF and VEGF

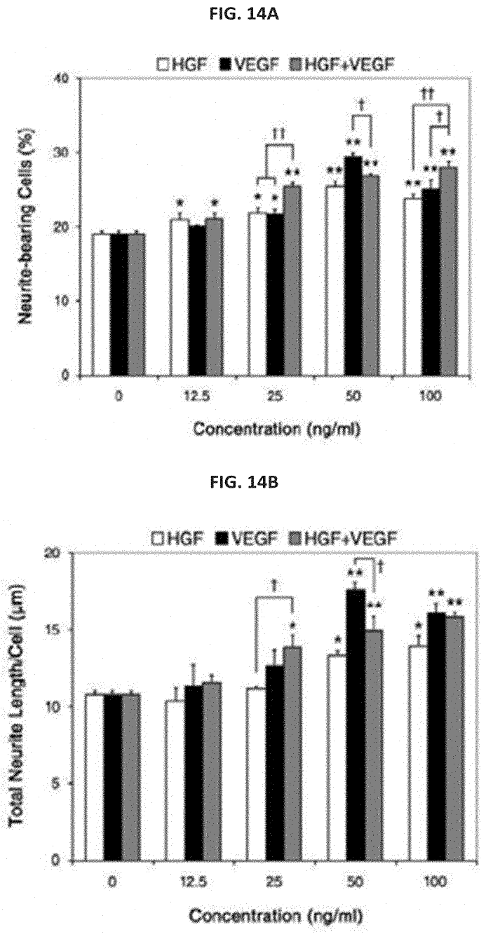

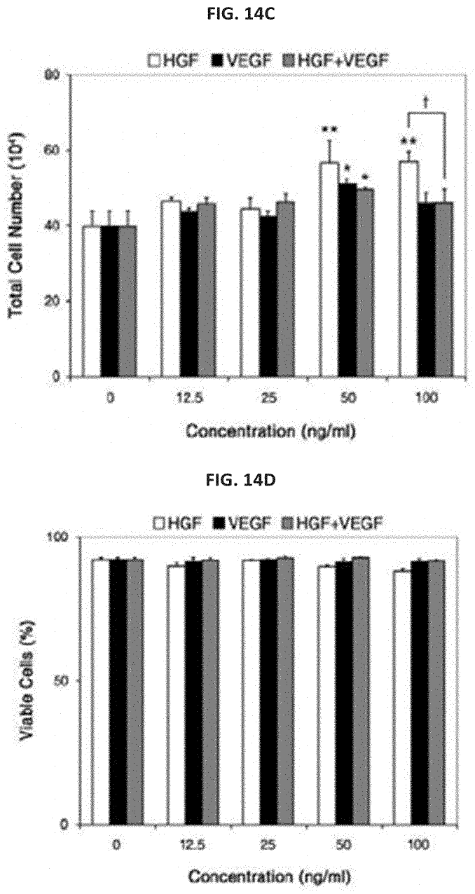

The present inventors cultured Neuro2A cells for 48 hours by adding 0 ng/mL, 12.5 ng/mL, 25 ng/mL, 50 ng/mL, and 100 ng/mL of recombinant HGF or VEGF protein (R&D Systems) to each cell medium, and the ratio of the cells having neurites with a length greater than the diameter of at least one cell body and the sum of the neurite lengths per cell in the Neuro2A cells obtained using a phase contrast microscope are shown in FIG. 14A and FIG. 14B, respectively.

As a result, as shown in FIGS. 14A and 14B, the Neuro2A cells, which were treated with 100 ng/mL HGF and 50 ng/mL VEGF, showed the highest growth in the length of neurites.

<8-2> Confirmation of Increase of Growth and Survival of Neurons by HGF and VEGF

The present inventors cultured Neuro2A cells for 48 hours by adding 0 ng/mL, 12.5 ng/mL, 25 ng/mL, 50 ng/mL, and 100 ng/mL of recombinant HGF or VEGF protein (R&D Systems) to each cell medium, stained the Neuro2A cells with trypan blue, and examined the effects of these proteins on the growth and survival of the cells. The total number of cells and the ratio of the survived cells are shown in FIG. 14C and FIG. 14D, respectively. The cell survival rate was measured as a percentage of the cells which were not stained with trypan blue (living cells). The above experiment was repeated independently at least three times and the results were indicated as mean±SEM.

As a result, as shown in FIGS. 14C and 14D, it was confirmed that exogenous HGF and/or VEGF proteins could increase the growth of neurites and cell growth of Neuro2A cells.

<8-3> Confirmation of Increase of Neurite Growth in Damaged Spinal Cord Tissue by HGF and VEGF

The nerve fibers of spinal cord tissue sections were stained with NF-M antibodies and Alexa 546 antibodies (red) in the same manner as in Example 7-1. On the 7^{th} day of culture, the spinal cord tissue sections were treated with lysolecithin (0.5 mg/mL) for 17 hours and cultured in a medium containing 0 ng/mL, 12.5 ng/mL, 25 ng/mL, 50 ng/mL, and 100 ng/mL of each of recombinant HGF or VEGF protein for a week. Then, the relative integrated optical density (IOD) values of the nerve fibers that were NF-M immunofluorescence stained were measured in the spinal cord tissue sections in the same manner as in Example 7-1, and the integrated optical density was normalized with the spinal cord tissue sections of the control group. The concentration unit (ng/mL) of the horizontal axis of the graph indicates the concentration for HGF alone, VEGF alone, or a mixed concentration for HGF+VEGF. The histograms represent mean±SEM for at least 5 tissue sections.

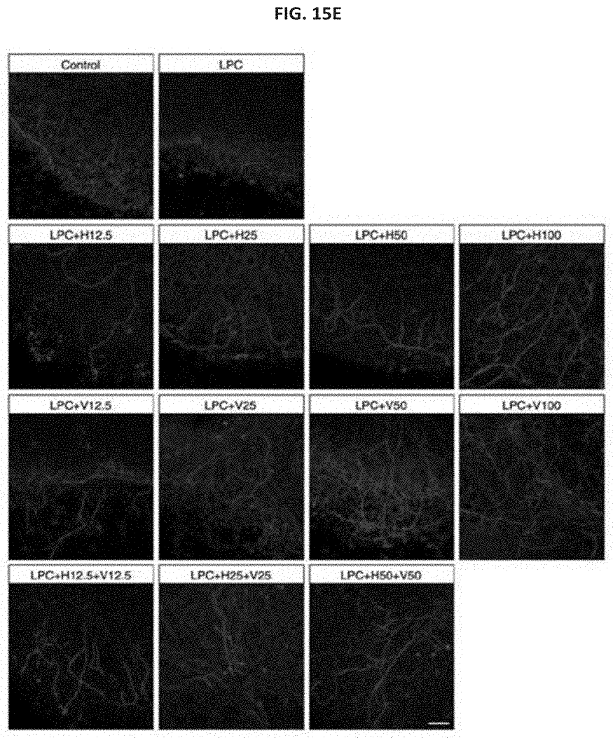

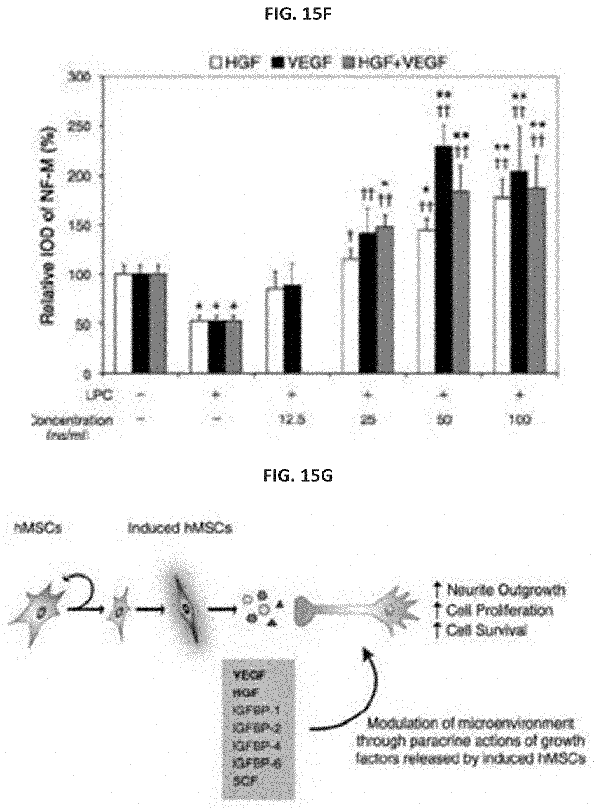

As a result, as shown in FIGS. 15E and 15F, it was confirmed that exogenous HGF and/or VEGF proteins could increase the neurite growth of damaged spinal cord tissue sections.

So far, specific embodiments of the present invention have been described with reference to the accompanying drawings, but it is obvious that the scope of the present invention include technical idea set forth in the following claims and their modifications and equivalents.

What is claimed is:

1. A cell composition comprising:
neural stem cells; and
neural stem cells derived from mesenchymal stem cells; and
at least one selected from the group consisting of immature neural stem cells and mesenchymal stem cells;
wherein the number of the neural stem cells is at least 70% of the number of the cells included in the cell composition, and the total number of the cells of the immature neural stem cells and the mesenchymal stem cells is at most 30% of the number of the cells included in the cell composition.

2. The cell composition of claim 1, wherein the cell composition further comprises neurons.

3. The cell composition of claim 1, wherein the cell composition further comprises GABAergic neurons.

4. The cell composition according to claim 1, wherein the mesenchymal stem cells are adipose tissue-derived mesenchymal stem cells or bone marrow-derived mesenchymal stem cells.

5. The cell composition of claim 1, wherein the neural stem cells and the neural stem cells derived from mesenchymal stem cells express at least one selected from the group consisting of Nestin, Sox2, Sox1, Musashi-1, FoxG1, Nkx2.1, Pax6, Gli3, Vimentin, Tuj1, Emx1, Olig2, and Ascl1.

6. The cell composition of claim 1, wherein the neural stem cells undergo a symmetric or asymmetric division.

7. The cell composition of claim 2, wherein the neurons express at least one selected from the group consisting of Tuj1, MAP2, FoxG1, Nkx2.1, Pax6, Dlx2, Dlx5, Lhx6, GAD67, SCN5A, Olig2, S100, and NeuN.

8. The cell composition of claim 3, wherein the GABAergic neurons express at least one selected from the group consisting of MAP2, Dlx2, Dlx5, GFAP, CALB2, GABRA1, GABRA2, GABRA5, GAD65, GAD67, PSD95, SYP, and NF-M.

9. The cell composition of claim 1, wherein the composition further comprises a cell growth factor.

10. The cell composition of claim 9, wherein the cell growth factors are epidermal growth factor, fibroblast growth factor, vascular endothelial growth factor (VEGF), hepatocyte growth factor (HGF), nerve growth factor (NGF), glial cell line-derived neurotrophic factor (GDNF), and platelet-derived growth factor (PDGF).

11. A neuron differentiated from a mesenchymal stem cell, wherein the neuron expresses at least one selected from the group consisting of Tuj1, MAP2, FoxG1, Nkx2.1, Pax6, Dlx2, Dlx5, Lhx6, GAD67, SCN5A, GFAP, S100β, Olig2, and NeuN.

12. A GABAergic neuron differentiated from a mesenchymal stem cell, wherein the GABAergic neuron expresses at least one selected from the group consisting of NKX2.1, LHX6, TuJ1, MAP2, MGE, Dlx2, Dlx5, GABRA1, GABRA2, GABRA5, CALB2, GAD65, GAD67, PSD95, NF-M, and SYP.

13. A composition for cell regeneration comprising:
at least one selected from the group consisting of neural stem cells, neurons, and
GABAergic neurons; and
cells that hypersecrete a growth factor.

14. The composition for cell regeneration of claim 13, wherein the composition for cell regeneration further comprises cells that express a marker expressed in at least one cell selected from the group consisting of astrocytes and oligodendrocytes.

15. The composition for cell regeneration of claim 13, wherein the neural stem cells express at least one selected from the group consisting of Nestin, Sox2, Sox1, Musashi-1, FoxG1, Nkx2.1, Pax6, Gli3, Vimentin, Tuj1, Emx1, and Ascl1.

16. The composition for cell regeneration of claim 13, wherein the cells that hypersecrete a growth factor express at least one selected from the group consisting of GFAP, P0, S100, CNPase, and p75NTR.

17. The composition for cell regeneration of claim 13, wherein the growth factor is at least one selected from the group consisting of hepatocyte growth factor (HGF), vascular endothelial growth factor (VEGF), insulin-like growth factor binding protein-1 (IGFBP-1), IGFBP-2, IGFBP-4, IGFBP-6, and stem cell factor (SCF), VEGF, and HGF.

18. The composition for cell regeneration of claim 13, wherein the composition for cell regeneration further comprises at least one selected from the group consisting of nerve growth factor (NGF), brain-derived neurotrophic factor (BDNF), neurotrophin-3 (NT-3), neurotrophin-4/5 (NT-4/5), neurotrophin-6 (NT-6), basic-FGF, acidic-FGF, FGF-5, epidermal growth factor (EGF), hepatocyte growth factor (HGF), insulin, insulin-like growth factor-1 (IGF-1), insulin-like growth factor-2 (IGF-2), glial cell line-derived neurotrophic factor (GDNF), TGF-β2, TGF-β3, interleukin-6 (IL-6), ciliary neurotrophic factor (CNTF), and LIF.

19. The pharmaceutical composition for improving or treating ischemic diseases comprising the composition for cell regeneration of claim 13.

20. The pharmaceutical composition for improving or treating neurological disorders comprising the composition for cell regeneration of claim 13.
